(19) United States
(12) Patent Application Publication
(10) Pub. No.: US 2006/0265776 A1
(43) Pub. Date: Nov. 23, 2006

(54) Viability

(76) Inventors: Antoni Ryszard Slabas, Durham (GB); Stephen Chivasa, Durham (GB); Bongani Kaiser Ndimba, Durham (GB); Keith Lindsey, Durham (GB)

Correspondence Address: MORGAN LEWIS & BOCKIUS LLP, 1111 PENNSYLVANIA AVENUE NW, WASHINGTON, DC, 20004, US

(21) Appl. No.: 10/551,718

(22) PCT Filed: Apr. 1, 2004
(86) PCT No.: PCT/GB04/01436

§ 371 Date: Jun. 23, 2006

(30) Foreign Application Priority Data
Apr. 1, 2003 (GB) 0307470.5

(51) Int. Cl. A01H 1/00; C12N 15/82; C12N 15/87

(52) U.S. Cl. 800/278; 800/285; 504/117

(57) ABSTRACT

Disclosed is a method of killing a plant cell or plant cells by activating a cell death pathway, which pathway is activatable by depletion of extracellular NTP, especially ATP, available to the cell for hydrolysis by cellular NTPase (especially ATPase) enzymes.

### FIELD OF INVENTION

This invention relates inter alia, to methods and compositions for controlling the viability of plant cells and, in particular, to methods and compositions for controlling the viability of plants, and especially to methods and compositions for killing plants or parts thereof.

### BACKGROUND OF INVENTION

List of abbreviations

The following abbreviations are used in this document:

ADP adenosine 5′-diphosphate

AMCD ATP-mediated cell death

AMP-PCP β,γ-methylenecidenosine 5′ triphosphate

AMP-PNP adenosine 5′-(β,γ-imido)triphosphate

ATP-γ-S adenosine 5′-(γ-thio)triphosphate

BVA biological variance analysis (module in Decyder software)

Cy2 C₃₂H₂₈N₃O₆C₂F₃O₂

Cy3 C₃₆H₄₄N₃O₄C₂F₃O₂

Cy5 C₃₆H₄₂N₃O₄C₂F₃O₂

DIGE difference in-gel electrophoresis

DMF dimethylformamide

GMP-PNP guanosine 5′-(β,γ-imido)triphosphate

GTP-γ-S guanosine 5′-(γ-thio)triphosphate

GTP guanosine 5′-triphosphate

IEF isoelectric focussing

MALDI-ToF matrix-assisted laser desorption ionization—time of flight

PMT photon multiplier tube

ATP is a ubiquitous, energy-rich compound that is found in all cells of free-living organisms. It is found both within organelles, such as mitochondria and chloroplasts, as well as in the cytoplasm. Glycolysis, oxidative phosphorylation and photophosphorylation are some of the cellular biochemical pathways capable of generating ATP. The energy from ATP is used to drive a number of essential biochemical reactions that are fundamental to the survival of cells and whole organisms. The presence of intracellular ATP has been recognised for a long time since its discovery in living cells (Fiske & Subbarow 1929 Science 70, 381-382).

Because of its molecular size and charge, ATP cannot cross the plasma membrane by simple diffusion and, therefore, would not normally be expected to occur extracellularly in the absence of cytolysis. However, two alternative mechanisms by which cells secrete ATP have been discovered. The first is exocytosis, a mechanism predominantly used by (but not exclusive to) nerve terminals where the released ATP functions as a neurotransmitter. The second mechanism utilises ABC transporters directly or by indirect activation of ATP channels. ATP release from animal cells was first reported in 1959 (Holton, 1959, J. Physiol (London) 145, 494-504).

The presence of extracellular ATP in a plant system was only recently recorded in Arabidopsis in 2000 (Thomas et al., 2000 Plant Cell 12, 519-533) and there are no reports of its occurrence in any other plant species. ATP extrusion in Arabidopsis is up-regulated in mutants over-expressing an ABC transporter (Thomas et al., 2000), suggesting that Arabidopsis employs this mechanism for export.

Further, Thomas et al teach that the ability to degrade extracellular ATP was important in resistance of plant cells to xenobiotics. Thus, Arabidopsis plants over-expressing an ecto-ATPase activity became more resistant than wild-type plants to xenobiotics, exemplified by cycloheximide. When an inhibitor of extracellular ATPase (α,β-methyladenosine 5′ diphosphate) was co-administered with the cycloheximide, the plants lost their cycloheximide-resistance.

Thomas et al also found that increasing the extracellular ATP concentration, by adding exogenous ATP, decreased the resistance of plants to xenobiotics. From this, those authors concluded that maintenance of an ATP gradient across the cell membrane was important for plant cell viability in the presence of a xenobiotic (i.e. low extracellular ATP concentration; high intracellular ATP concentration), and suggested that plant cells use such an ATP gradient to help power the efflux of toxic compounds from the cell by efflux proteins, such as P-glycoprotein.

Thomas et al did not teach or suggest that phosphatase inhibitors might cause plant cell death in their own right, in the absence of a xenobiotic. Indeed, to the contrary, Thomas et al taught that decreasing the extracellular ATP concentration might enhance plant cell viability, by making the ATP gradient across the plasma membrane steeper. Substantially similar findings and teachings were reported by Windsor et al (WO 01/64859).

Controlling cell death/viability has long been important in the development of targets for new herbicides. Selective cell viability is also important as a way of altering development of plants by causing death of important tissues or particular cell lineages. This has application in male-sterility in plants and possibly altering developmental morphology of entire organisms. In addition, delaying cell death can extend the longevity of plants and this can be of commercial importance. For example, delaying flower and leaf abscission by controlling cell death can potentially increase the “shelf-life” of ornamental plants. Moreover, prevention of flower and fruit abscission automatically increases the yield of crop plants. The control of cell viability is also important in disease control. Various treatments have been devised which use specific delivery systems for killing particular cell types. The inventors herein demonstrate that cell death, in the absence of an exogenous xenobiotic, can be mediated by reduction of extracellular ATP levels and/or by preventing its hydrolysis by cellular enzymes. This has utility in identifying new herbicides and control of organism development.

All publications mentioned in this specification are specifically incorporated herein by reference.

### SUMMARY OF INVENTION

The present invention provides a novel way of killing plant cells and whole plants or parts thereof by, for example, depleting the amount of extracellular NTP (nucleotide triphosphate), especially ATP, available to plant cells. The invention allows one to select/identify new herbicides, novel strategies to control diseases, and the control of cellular or whole plant morphology. It is demonstrated that suspension cultures of Arabidopsis and corn (Zea mays) are killed when extracellular ATP is removed or competitively excluded from its binding sites, by (i) incubation with glucose and hexokinase, which utilises ATP to generate glucose-6-phosphate, or (ii) incubation with apyrase, an enzyme that hydrolyses ATP to AMP and inorganic phosphate in a 2-step reaction with ADP as an intermediate product. The inventors have also found that addition of non-hydrolysable analogues of nucleotide triphosphates provides a means of killing cells by effectively decreasing the level of extracellular NTP available to enzymes by competing for binding to active sites and so excluding NTP from participating in essential biochemical processes. Mechanisms that result in lowering the concentration of extracellular ATP or rendering it non-available to the plant cell (e.g. prevention of its utilization by cellular enzymes) can thus be used to mediate cell death. This can be done in a variety of different ways, as described more fully below.

Conversely, the inventors have found that in conditions in which extracellular ATP is undesirably depleted or in which undesirable depletion of extracellular ATP is triggered (e.g. by the presence of pathogens), then cell viability can be enhanced or improved by addition of exogenous ATP.

The inventors believe that a similar effect may result following depletion of the extracellular concentration of one or more of the other naturally-occurring nucleotide triphosphates (CTP, GTP, TTP), especially GTP. Whilst it is preferred that the invention concerns alteration of the extracellular concentration of ATP, it is possible that the same or a similar pathway is activated by depletion of the other nucleotide triphosphates (NTP).

Thus, in general terms, the invention provides a method of controlling the viability of a plant cell or cells by contacting the cell or cells with a substance which directly or indirectly up- or down-regulates a cell death pathway in the cell or cells, which pathway is activatable by depleting the concentration of NTP, especially ATP, in the extracellular environment available to the cell or cells. By depleting extracellular NTP, or triggering such a depletion, the viability of the cell or cells can be abolished whilst, for a cell or cells exposed to extracellular NTP depletion, the viability can be preserved by, for example, providing exogenous NTP, especially exogenous ATP.

More specifically, in one aspect the invention provides a method of killing a plant cell by activating a cell death pathway, which pathway is activatable by depletion of extracellular NTP, especially ATP, available to the cell for utilization (e.g. by extracellular NTPase enzymes).

In this context “extracellular NTPase enzymes” is intended to encompass enzymes which are secreted or otherwise exported to the exterior of the cell or are present on or in the cell membrane (and includes multi-activity enzyme complexes which possess an NTPase activity), such that NTP which is extracellular, or is of extracellular origin, is an available substrate for the enzyme. Note also that NTP binding to a particular receptor (rather than its hydrolysis by an NTPase) may be sufficient to modify the viability of the cell.

“Activating” the cell death pathway means triggering the pathway in some manner such that, after activation, the activity of one or (preferably) more enzymes, which catalyse particular reactions in the pathway, is increased. The activity may increase by accumulation of greater amounts of the enzyme/s in question and/or by conversion of the enzyme from a relatively inactive form to a relatively active form (e.g. by dephosphorylation). The pathway may be activated at an upstream position (e.g. by depletion of extracellular NTP, especially ATP) and/or at one or more intermediate positions downstream of extracellular NTP depletion.

In classical biochemical terms, a pathway may typically be activated by increasing the concentration of a substrate of the pathway and/or by depleting the effective concentration of the product/s of the pathway.

The plant cell whose viability is to be controlled may be in culture in vitro or may be part of a plant or plantlet. Thus, in a particular embodiment the invention provides a method of killing a plant or part thereof, by killing a plurality of the cells within the plant or part thereof to be killed. Those skilled in the art will appreciate that it is not necessary to kill all the cells of a plant or part thereof in order to kill the plant or part thereof (as appropriate)-killing, for example, the majority of the cells will normally render the remainder non-viable within a limited timescale (e.g. typically within 10 days, preferably within 7 days, more preferably within 5 days, and most preferably within 3 days).

Equally, whilst activation of the extracellular NTP-depletion mediated cell death pathway can be used to abolish plant cell viability, inhibition of the pathway at one or (preferably) more points will act to preserve viability of plant cells in conditions of extracellular NTP-depletion or other circumstances which would tend to trigger cell death. The inventors can envisage a number of ways of inhibiting the cell death pathway. Typically a pathway may be inhibited or down-regulated by the use of a substance which utilises components of the pathway in such a way as to divert the cell death “signal”. The substance may be, for example, a reversible or irreversible inhibitor of one or more enzymes in the pathway. Classically, such inhibitors may be a structural analogue of the enzyme's intended substrate and thereby prevent the enzyme acting on its intended substrate, in a competitive or non-competitive manner.

An alternative approach would be to cause over-expression of polypeptides in the cell death pathway which are altered in some way (e.g. mutated) so as to render them inactive (in the sense of being unable to propagate the “cell death signal” along the pathway), then such inactive polypeptides would compete with the plant cell's intrinsic active proteins and effectively swamp them. Such altered polypeptides could of course be expressed in the plant cell by genetic modification, e.g. introducing a nucleotide sequence into the plant cell which expresses the altered polypeptide at high concentration.

In a further aspect the invention provides a composition for controlling the viability of a plant or plant cell, the composition comprising an active agent which, directly or indirectly, up- or down-regulates in the plant cell or cells, a cell death pathway, which pathway is activatable by depletion of extracellular NTP, especially ATP, available for utilization by the cell or cells. The composition will advantageously comprise other constituents conventionally present in herbicidal formulations, and which will be well known to those skilled in the art, such as surfactants and penetration enhancers, (see, for example, Brand & Mueller 2002, Toxicological Sciences 68, 18-23, and references cited therein).

Typically the composition will be made and sold as a concentrate, which must be diluted with water or other diluent before use.

One way of activating the relevant cell death pathway discovered by the inventors is to cause depletion in the extracellular environment of the level of NTP, especially ATP, available for hydrolysis or other utilization by the plant cell. This may be done by actually removing or destroying extracellular NTP and/or may be achieved by otherwise rendering that extracellular NTP which is present non-available to the cell.

Preferably the method of the invention involves the step of bringing an active agent into contact with the extracellular environment of the cell or cells to be killed, which agent has the effect of hydrolysing extracellular NTP (especially ATP) and/or rendering extracellular NTP (especially ATP) non-available to the plant cell or cells, or otherwise activating the cell death pathway.

Agents which hydrolyse NTP include apyrases, or kinases, such as hexose kinase (preferably in combination with a suitable phosphate group acceptor substrate, e.g. hexose kinase in combination with hexose, especially glucosekinase in combination with glucose).

Agents which render, for example, extracellular ATP non-available to the plant cell include compounds which bind to ATP and prevent its uptake or use by the plant cell. Other compounds which render the extracellular ATP non-available to the plant cell include compounds which are competitors of ATP i.e. substances which will bind (preferably with an affinity equivalent to or greater than that of ATP), to ATP-binding sites on the exterior of the cell. (A competitor which has a lower binding affinity than ATP may nevertheless be effective if it can be provided at a concentration which effectively swamps any extracellular ATP present.) Such ATP-binding sites will typically be present on kinases or other ATP-hydrolysis-linked enzymes. In particular, the competitor compound may be an analogue of an NTP, especially an analogue of ATP which is non-hydrolysable (i.e. cannot be hydrolysed by those enzymes produced by the plant which are capable of hydrolysing ATP), such that once the competitor has occupied the ATP-binding site on the exterior of the cell it will essentially or substantially prevent, or at least inhibit, ATP being subsequently bound.

It will be apparent to those skilled in the art that it is not necessary for an analogue to be completely non-hydrolysable in order for it to have a lethal effect. For example, if an analogue is hydrolysed by a particular plant extracellular ATPase (or “ectoATPase”) only slowly (compared to the rate of hydrolysis of ATP), then it may still act as an effective inhibitor. In particular, if the analogue is present in a concentration which is much higher than the extracellular ATP concentration, the analogue may compete effectively. Thus, ATP analogues which are hydrolysed at rates up to about 30% of the rate of hydrolysis of ATP may still be useful in the present invention, and the term “non-hydrolysable” should accordingly be broadly construed where the context permits. Rates of enzymatic catalysis can readily be determined by those skilled in the art by use of conventional biochemical techniques (e.g. colourimetric assays and the like).

A number of ATP analogues (of varying ease of hydrolysis by ATPases), and/or inhibitors of ectoATPases are known, including:

phosphorothioate analogues (such as ATPγS and ADPβS); phosphonate analogues (such as AMP-PCP and AmP-PNP); guanidyl and uridyl equivalents of the foregoing; ATP, GTP and UTP analogues comprising one or more substitutions on the purine ring (e.g. at position C2 or C8); RNA aptamers (see, e.g., Vaish et al, 2003 Biochem. 42, 8842-8851); bicyclic pyrimidine derivatives (Makara et al, 2001 J. Org. Chem. 66, 5783-5789); amidoferrocenyl- and pentamethylamidoferroceryl dendrimers (Daniel et al, 2003 Chemistry—A European Journal 9, 4371-4379). Still others are described by Bagshaw (2001 J. Cell Sci. 114, 459-460) and Gendron et al, (2000 Adv. Exp. Med. Biol. 486, 119-123; 2000 J. Med. Chem. 43, 2239-2247; and 2002 Curr. Drug Targets 3, 229-245). Many of these compounds are commercially available from standard sources (e.g. Sigma/Aldrich, Calbiochem, Boehringer etc.).

In addition there are known inhibitors of ATPsynthase which may well have inhibitory effects on plant ectoATPases. These include aluminium fluoride (Menz et al, 2001 Cell 106, 331-341; Braig et al, 2000 Struct. Fold Des. 1,567-73), DCCD (Gibbons et al, 2000 Nat. Struct. Biol. 7, 1055-1061), efrapeptin (Abrahams et al, 1996 Proc. Natl. Acad. Sci. USA 93, 9420-4) and the inhibitory peptide IF₁ (Cabezon et al, 2001 EMBO J. 20 6990-6996; van Raaij et al, 1996 Biochemistry 35, 15618-15625), 4-chloro-7-nitrobenzofurazan (Orriss et al, 1998 Structure 6, 831-837); 4-azido-2-nitrophenyl phosphate (Groth et al, 2000 Biochemistry 39, 13781-7); aurovertin B (van Raaij et al, 1996 Proc. Natl. Acad. Sci. USA 93, 6913-6917); and tentoxin (Minoletti et al, 2002 Proteins 49, 302-320).

The 3D structure of ATPase is known from X-ray crystallographic studies. It should be possible therefore, using conventional commercially available, computer modelling programs, such as GRAM, DOCK and AUTODOCK (Walters et al, 1998 Drug Disc. Today 3, 160-178; Dunbrack et al, 1997 Folding and Design 2, 27-42) rationally to design other compounds which might be expected to bind to ectoATPases.

Alternatively a computer program may be employed to analyse the active site of ATPase and predict the structure of chemical moieties which will interact with the active site. An example of one such program is GRID (Goodford et al, 1985 J. Med. Chem. 28, 849-857).

Equally, it should be possible rationally to design other molecules, based on the structure of known ATPase inhibitors and/or non-hydrolysable analogues of ATP, which have closely similar overall shape and charge profiles, and which might also therefore be predicted to inhibit ATPase.

Compounds and molecules designed in this way can then be synthesised and variants made in a chemical combinational library and the library constituents tested in vitro in high throughput screens for inhibitory activity. Those compounds exhibiting ATPase inhibitory activity can then be further investigated e.g. for herbicidal activity in plants and for human safety etc.

Those skilled in the art will appreciate that the cell death pathway which is activatable by the depletion of extracellular NTP available for utilization may equally be activated (or inhibited) at a point downstream of NTP depletion. For example, the inventors provide evidence (below) that extracellular ATP depletion results in the dephosphorylation of several plant cell polypeptides. It may be hypothesised that one or more of these polypeptides must be in phosphorylated form in order to retain a desirable biological activity, such that dephosphorylation will eventually kill the plant cell. Detailed genomic and proteomic studies, along the lines of those described below in the Examples, and within the capability of those of normal skill in the art, reveal the changes which take place in the cell at the polypeptide level when the cell death pathway is activated, such that other methods of, and agents for, activating the pathway will be available to those skilled in the art.

In particular, the inventors have identified 59 polypeptides, the level of expression of which is significantly altered following various treatments which deplete or increase the concentration of extracellular ATP, and which are therefore potential candidate targets for methods and compositions for controlling the viability of a plant cell or cells. These target polypeptides are identified in Table 3, and in Appendix 1, below.

Accordingly, by inhibiting or “activating”, as appropriate, one or more (preferably at least two) of the polypeptides identified in Table 3, it should be possible to up- or down-regulate the viability of a plant or part thereof.

The relevant polypeptides could be “activated”, for example, by increasing the level of expression in the plant. This would typically be accomplished by genetic manipulation (e.g. producing plants which comprise additional copies of the relevant gene, and/or by inserting more efficacious promoters and/or enhancers in operable combination with the relevant genes).

Selected polypeptides could be inhibited in any of a number of ways. In one embodiment, a suitable inhibitor (preferably one which is a specific inhibitor of the relevant polypeptide/s) may be administered to the plant or part thereof. For example, some of the polypeptides identified as being implicated in AMCD are: vacuolar ATP synthase (NCBI accession no. gi 2493132); alanine aminotransferase (NCBI accession nos. gi 21954069, gi 21954071 and gi 9082270); glutathione S transferase (NCBI accession nos. gi 15224581 and 2, gi 15218639 and 40); and thioredoxin. Known inhibitors of vacuolar ATP synthase include the antibiotics Bafilomycin A and Concanamycin A. Known inhibitors of alanine aminotransferase include amino oxyacetate. Known inhibitors of glutathione S transferase include 2,3-dichloro-4-(2-methylenebutyryl)-phenoxyacetic acid (or a “ethacrynic acid”), and (Z)-3-benzyllidene-3,4-dihydro-2-oxo-2H, 4-benzoxazine-carboxylic acid. Known inhibitors of thioredoxin include cis-diamminedichloroplatinum (also known as cisplatin) and 1-methylpropyl 2-imidazolyl disulfide.

In addition to the foregoing, many inhibitors of ATPases are known (as mentioned elsewhere) and one or more of these should be effective in inhibiting the ATPases identified in Table 3.

Thus, in a further aspect, the invention provides the use of an ATP analogue, or an ATPase inhibitor, as an active agent in the preparation of a herbicidal composition.

In another aspect, the invention provides the use of an inhibitor or other antagonist of any one of the polypeptides listed in Table 3, as an active agent in the preparation of a composition to modulate the viability of a plant or part thereof.

Another approach to inhibiting the action of any one or more of the polypeptides listed in Table 3 would be to use genetic manipulation. For example, the amino acid sequence of the proteins is known, and the corresponding nucleotide sequence of the nucleic acid encoding the polypeptides.

A recombinant nucleic acid molecule comprising a sequence of at least 200 bases (preferably at least 300 bases, more preferably at least 400 bases) having at least 90% sequence identity with a sequence encoding one of the polypeptides listed in Table 3, may be of use in putting the invention into effect. The sequence may preferably be operably linked in either the sense or antisense orientation, as desired, to a suitable promoter active in the plant.

Plant promoter sequences are well known to those skilled in the art and include both constitutive and inducible promoters. Examples include the CaMV 35S promoter, the RUBISCO small subunit (SS) promoter, and the Chlorophyll alb binding protein promoter. A list of plant promoters is available from the “Plant Prom” database (Bioinformatics Web Server, Royal Holloway College, University of London, Department of Computer Science). Equally nucleic acid constructs suitable for use in plants are well known to those skilled in the art and do not require detailed description. Methods of introducing nucleic acids into plant cells or whole plants are also well-known to those skilled in the art and include: electroporation; microinjection; transduction; Agrobacterium-mediated transformation or use of Ti plasmid-based vectors; and protoplast transformation.

The composition of the invention may be selectively applied e.g. by injection or surface application to particular parts of plants to cause cell death restricted to desired portions. Alternatively, substantially all of the plant may be exposed to the agent (e.g. by spraying the composition onto the plant), to cause the death of the whole plant.

The inventors have found that exposure of plant cells to bacterial pathogens (as exemplified by Ps. syringae) or fungal pathogens (as represented by Fusarium elicitor substance), can cause cell death by a mechanism which involves depletion of extracellular NTP, and that the viability of plant cells exposed to these agents can be restored to near normal levels by causing an increase in NTP concentration in the extracellular environment of the cells. Thus, in a particular further embodiment within the overall concept of the invention, there is provided a method of preserving the viability of a plant cell or cells exposed to a viability-threatening depletion of extracellular NTP (whether triggered by presence of pathogens or other causes), the method comprising the step of administering a viability-preserving substance which has the effect of increasing the extracellular NTP concentration or otherwise inhibiting the cell death pathway which has been activated (e.g. by a pathogen). Where the method is performed in vitro, the viability-preserving substance may simply be introduced into the culture medium. Where the method is performed in planta, the viability-preserving substance may conveniently be introduced into the plant by spraying onto the surface thereof, or by application in solution to the soil or other water-source of the plant, or less preferably by direct injection into the plant. Most simply, the viability-preserving substance comprises NTP (especially ATP) but may be any substance which has, as a result of interaction with one or more other substances present in the plant cell and/or in the extracellular environment, the effect of augmenting the extracellular NTP (especially ATP) concentration or otherwise inhibiting the cell death pathway. This method of the invention may find particular usefulness in protecting a plant against attack by pathogens.

It might also be possible to produce new plant varieties by conventional breeding or by genetic manipulation/modification, for example, introducing a gene or genes encoding for polypeptide(s) which inhibit or counteract extracellular NTP (especially ATP) depletion caused or triggered by pathogens, or otherwise inhibit the cell death pathway, and thereby render the new variety resistant to cell death e.g. caused or triggered by certain pathogens. For instance, the viability of a plant cell could be preserved by introducing nucleotide sequences to encode the components of a biochemical pathway which result in elevated levels of extracellular NTP (especially ATP) either constitutively or specifically in response to an event (such as attack by a pathogen) which tends to deplete extracellular NTP (especially ATP). Such sequences and/or the polypeptides encoded thereby can be regarded as a viability-preserving substance.

As mentioned above, Table 3 lists specific polypeptides identified by the inventors as involved in the phenomenon of ATP-mediated cell death (AMCD). Accordingly, inhibiting one or more relevant peptides might enhance or decrease the viability of a plant or part thereof in a particular situation.

Accordingly, in a further aspect, the invention provides for use of a recombinant nucleic acid molecule comprising a sequence of at least 200 bases (preferably at least 300 bases, more preferably at least 400 bases) having at least 90% sequence identity with a sequence encoding one of the polypeptides listed in Table 3, operably linked in the sense or antisense orientation to a promoter active in a plant, in the preparation of a composition to alter the viability of a plant or part thereof; and a corresponding method.

The invention also provides a plant or part thereof having altered viability resulting from the introduction of a nucleic acid molecule as defined above.

Preferably the use, method and altered plant involves the use of two or more sequences, each of at least 200 bases etc. and each having at least 90% sequence identity with a different sequence encoding a respective different polypeptide listed in Table 3. The two or more sequences may be present on different nucleic acid constructs or present on the same construct. If present on the same construct, the two sequences may be operably linked to a single, common promoter or to respective separate promoters (which may be identical or different).

The inventors believe that the AMCD phenomenon they have discovered may be triggered in a plant by a wide range of different stimuli or stresses (e.g. dehydration, pathogen attack, nutrient deficiency etc.). Thus, for example, the invention provides in a particular embodiment, a transgenic plant having up- or down-regulated responsiveness to stress and/or altered viability, as a result of the introduction of one or more nucleic acid molecules as referred to above.

The introduced sequence may direct the expression a full length or active polypeptide, so as to increase the concentration of the polypeptide in the plant or part thereof. Alternatively, the introduced sequence may direct the synthesis of a transcript which has an inhibitory effect on the expression of an endogenous gene present in the plant (e.g. as a result of antisense or RNA: interactions).

The invention will now be further described by way of illustrative example and with reference to the accompanying drawings, in which:

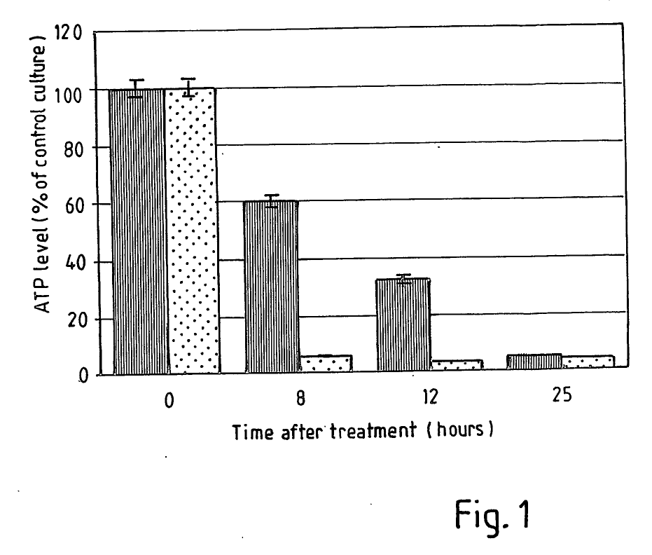

FIG. 1 is a bar chart showing ATP level (expressed as a % of levels in control cultures) after various durations (in hours) of ATP depletion treatments. The error bars show the standard deviation;

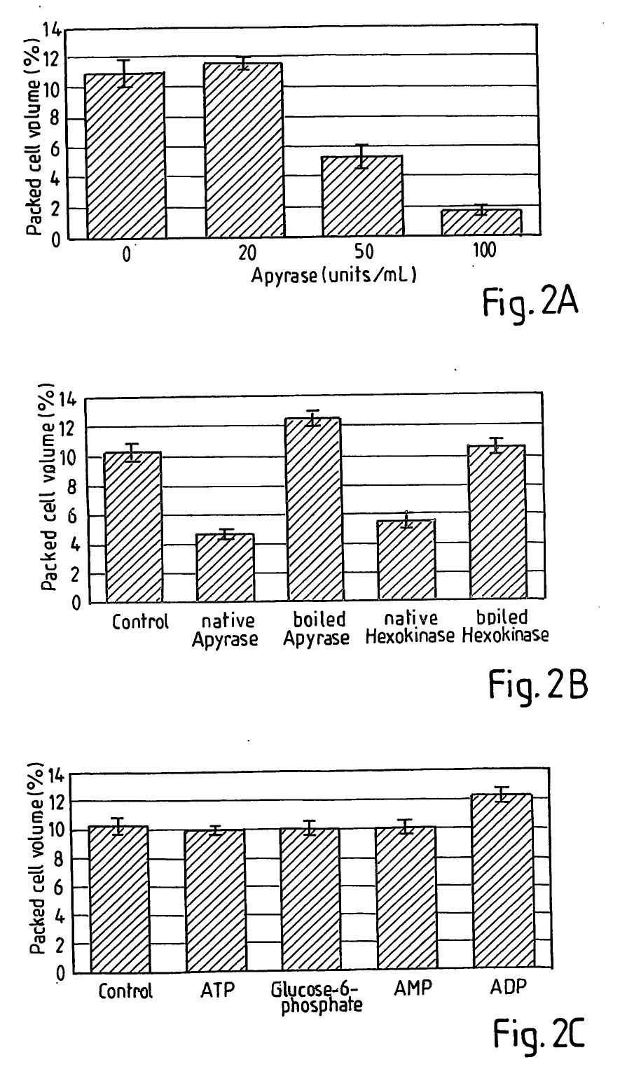

FIGS. 2A-C and 3A-C are bar charts showing cell viability (expressed as packed cell volume %) for plant cell suspension cultures subjected to control or various experimental treatments. The error bars show the standard deviation;

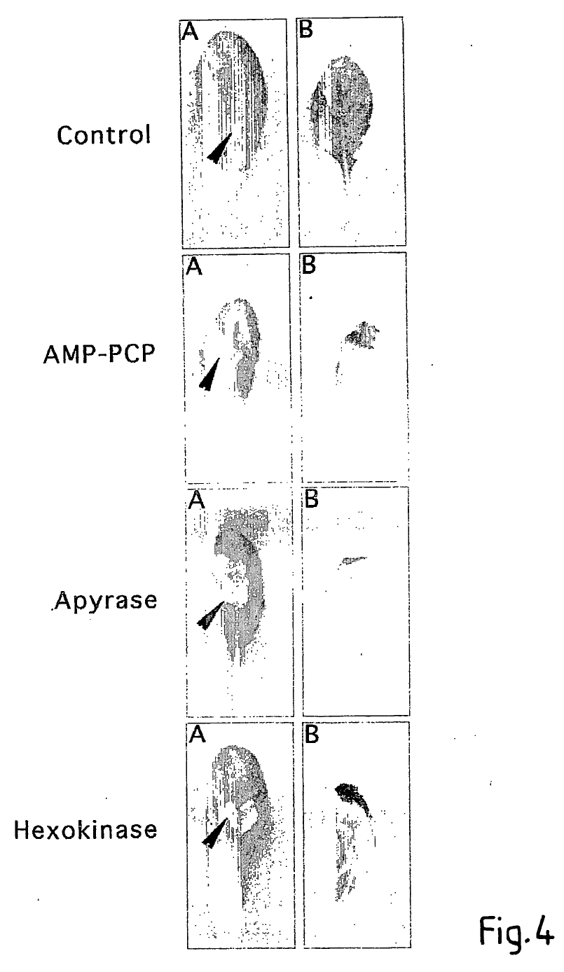

FIGS. 4A, B are photographs of plant leaves showing the effects of control or experimental treatments on various plants;

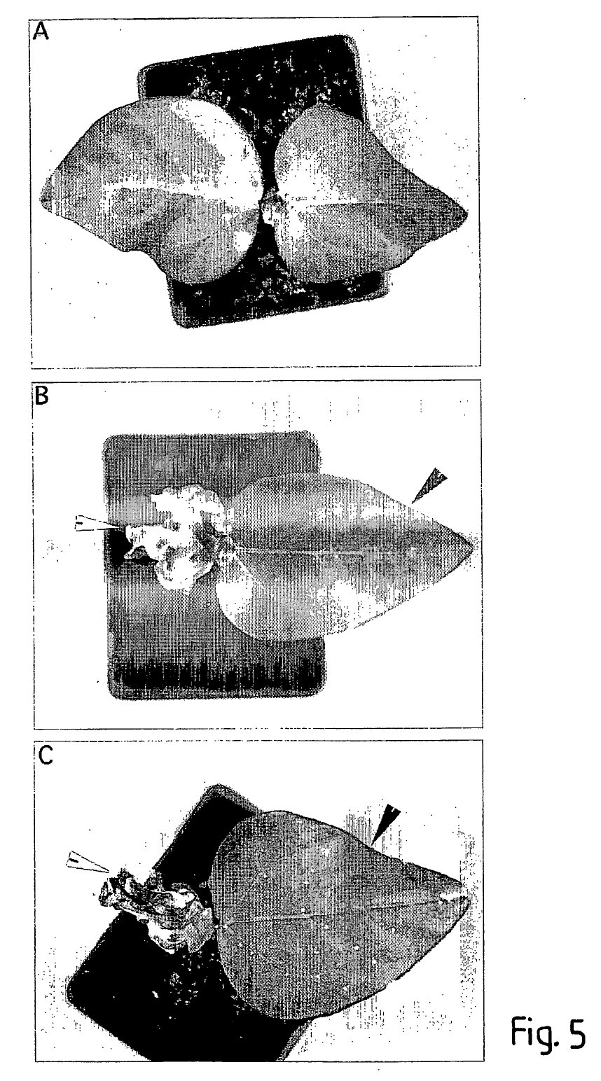

FIGS. 5A-C and 6A,B are photographs of plants showing the effects of control or experimental treatments on various plants;

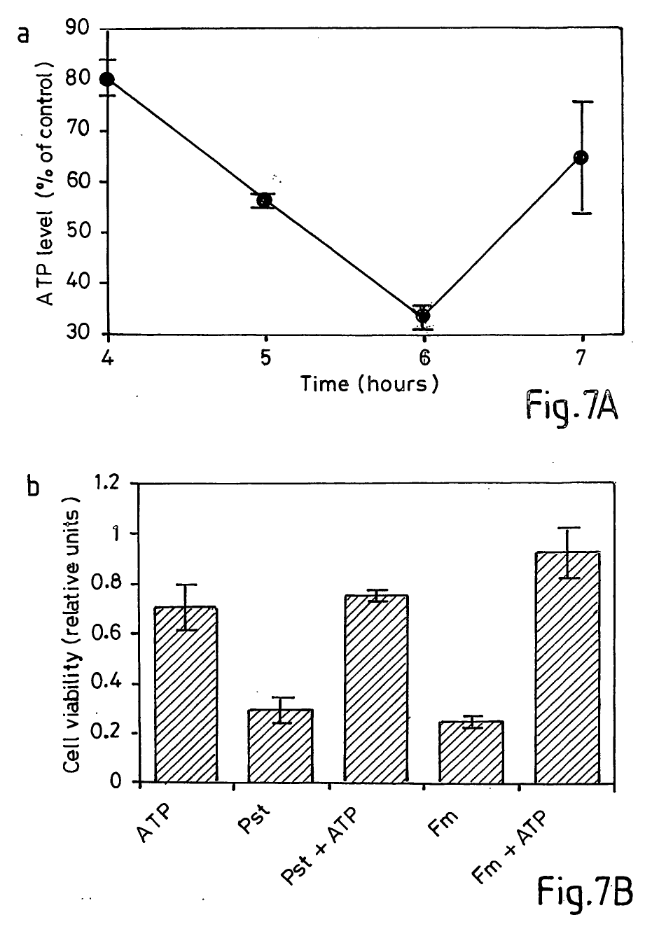

FIG. 7A is a graph of % extracellular ATP level against time (in hours); and

FIG. 7B is a bar chart showing cell viability (arbitary relative units) for Arabidopsis cultures 24 hours after various treatments.

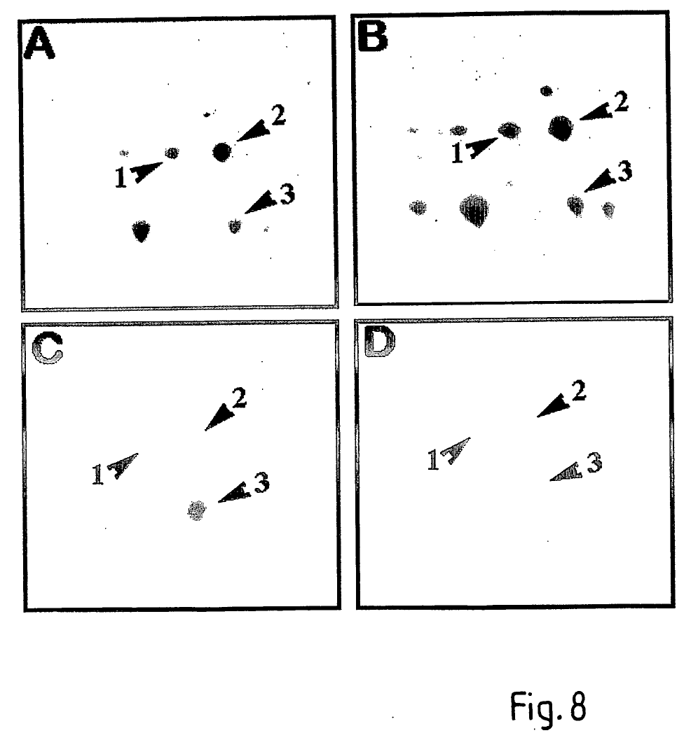

FIGS. 8A-D are photographs showing the results of 2D-gel electrophoresis analysis of extracellular proteins of A. thaliana cultures;

### EXAMPLES

### Example 1

### Treatment of Arabidopsis thaliana Cell Suspension Cultures with Apyrase or Hexokinase/Glucose Removes Extracellular ATP

A suspension of Arabidopsis thaliana cells was grown in MS medium Murashige & Skoog, 1962 Physiol. Plant. 15, 473497) with minimal vitamins and containing 3% (w/v) sucrose, 0.5 mg/L kinetin, and 0.5 mg/L 1-naphthalene acetic acid, and adjusted to pH 5.7 with NaOH/HCl. All the medium components were purchased from Sigma Chemical Company (Poole, UK). The culture was propagated by weekly sub-culturing of 7 day old inoculum into fresh medium (10-fold dilution) and incubating on a rotary platform (125 r.p.m) at 25° C. in complete darkness. Such cells are viable for many days after transferring to fresh growth medium. Cells were normally grown as 100 mL cultures in 250 mL glass Erlenmeyer flasks, but all treatments were performed on 1.5 mL or 10 m/L aliquots in sterile plastic vials of 3.5 cm diameter (Bibby Sterilin Ltd., Stone, UK). Cell cultures were used for treatments 3 days after inoculating fresh medium.

A final concentration of 100 nM glucose (filter sterilized) was added to a 3 days old cell suspension culture that was then divided into 10 mL aliquots. The aliquots were treated with a final concentration of 100 units/mL apyrase or 200 units/mL hexokinase. Both apyrase and hexokinase (Sigma Chemical Co.) were dissolved in deionised water and filter-sterilised using 0.2 μm filters. Control cultures were treated with an equivalent volume (2 ml) of sterile deionised water. The cultures were incubated for a total of 25 hours, with 500 μL aliquots of culture medium being withdrawn for ATP assays at 0, 8, 12, and 25 hours after treatment. The 500 μL aliquots were mixed with 10 μL of 50% (w/v) trichloroacetic acid containing 0.0005% (w/v) xylene cyanole FF and immediately frozen in liquid nitrogen. These samples were then thawed and the precipitated protein pelleted and discarded. For ATP assay, 5 μL of the supernatant were mixed with 95 μL of 100 mM Tris-acetate buffer (pH 7.8) in wells of non-transparent 96-well microtitre plates. The assay was performed on duplicate samples by adding 30 L of a luciferin/luciferase mix reagent (Promega, Southampton, UK) followed by a reading delay of 0.3 seconds and an integration time of 2 seconds. The 30 μL of luciferin/luciferase mix were applied via an automatic reagent feeding line fitted to the luminometer (model Anthos Lucy 1; Labtech International Ltd., Ringmer, UK). Water and fresh growth medium were used as blanks.

The amount of extracellular ATP in the treated cultures was expressed as a percentage of extracellular ATP in the control cultures at each time point. FIG. 1 is a time-course of extracellular ATP levels in Arabidopsis cell suspension cultures treated with apyrase (gray blocks) and hexokinase (spotted blocks) as a percentage of the extracellular ATP in control cultures. Error bars represent the standard deviation. The figure shows that both apyrase and hexokinase had reduced the amount of extracellular ATP levels below 5% of the amount in the control cultures within 25 hours of commencing treatment. Hexokinase phosphorylates glucose to glucose-6-phosphate by transferring a phosphate group from ATP, and thus producing ADP. Apyrase dephosphorylates ATP to ADP and ADP to AMP. These reactions consume ATP and account for the observed reduction of extracellular ATP attending the treatment of cell cultures with both enzyme systems. However, hexokinase was more rapid in reducing the level of extracellular ATP in this system than apyrase, as it reduced extracellular ATP to levels less than 5% within 8 hours of treatment. This is because the intermediate product of ATP catabolism by apyrase is also a substrate for this enzyme, hence the rate of ATP dephosphorylation decreases as more ADP is produced These results demonstrated that apyrase and hexokinase activities are sufficient to effectively reduce the amount of extracellular ATP in a plant cell suspension culture system.

### Example 2

### Removal of External ATP by Treatment of A. thaliana Cell Suspension Cultures with Apyrase Results in Cell Death

A. thaliana cell cultures were grown as described in example 1. Aliquots of the cell suspension (1.5 mL) were treated with a final concentration of 0, 20, 50, or 100 units/mL of apyrase. The cultures were incubated for 3 days and the apyrase-treated cultures showed a significant frequency of cell death. The dead cells had become buoyant and adhered to the walls of the vials, forming a ring just above the edge of the swirling medium. The ring of cells was sometimes dislodged and fell into the medium, resulting in apyrase-treated cultures having flakes of dead cells at the bottom of the vials.

The inventors decided to use viability staining to confirm cell death in these cell cultures. To achieve this, 200 L aliquots were removed from the cultures and the cells resuspended in 0.2M CaCl₂ after removal of the growth medium. The aliquots were doubly-stained by incubating for 5 minutes with a final concentration of 25 μg/mL fluorescein diacetate [0.5% (w/v) stock solution in acetone] and 50 μg/mL propidium iodide [10 mg/mL stock solution in phosphate-buffered saline pH 7.4]. Microscopic examination under UV light revealed live cells, which were emitting green fluorescence. The viable cells had also excluded propidium iodide, which is non-permeative and can only cross membranes of dead or dying cells. Dead cells had taken up propidium iodide, which binds to DNA, resulting in nuclei emitting a very intense red fluorescence. The inventors observed that a significantly high proportion of cells in the apyrase-treated cell cultures was dead and it was confirmed that the flakes of cells found at the bottom of these cultures were indeed dead.

To quantify the effect of apyrase on cell viability, the ability of the cells to grow and multiply subsequent to treatment was measured after diluting the cultures by over 30 times in fresh medium not containing any additives. This was achieved by transferring the cells treated for 3 days to 50 mL of fresh growth medium and allowing them to grow for a further 4 days. At the end of this period, triplicate 1 mL aliquots were sampled from the cultures and the volume of the cells was measured and expressed as a percentage of the culture volume. The method used to determine the packed cell volume was as follows: 1 mL cell culture aliquots were placed in 1.5 mL microfuge tubes and the cells gently compacted by centrifuging (1000 rpm., 10 minutes) in a swing-out rotor (Grundrotor 11030; Sigma laborzentrifugen GmbH, Oestrode, Germany). The level of the cells was marked on the wall of the tube and the volume of cells determined by measuring the volume of water needed to fill the tube to the marked level. The packed cell volume was expressed as the volume occupied by cells as a percentage of 1000 μL.

FIG. 2A shows the dose-response of Arabidopsis cells to treatment with apyrase at 0, 20, 50 or 100 units/ml. FIG. 2B shows the results obtained when using native or boiled apyrase and glucose (100 mM) in combination with native or boiled hexolinase (apyrase at 50 units/ml, hexokinase at 200 units/ml). FIG. 2C shows the effects of treating the cells with either ATP (11 mM) or with glucose-6-phosphate (100 mM), AMP(1 mM) or ADP (1 mM). In each case the error bars represent the standard deviation.

As apparent from FIG. 2, increasing apyrase concentration caused a progressive decrease in cell viability with over 80% loss in viability at 100 units/mL apyrase. When apyrase was denatured by boiling for S minutes prior to treating the suspension cultures, the treatment did not result in cell death (FIG. 2B) revealing that the cell death resulting from treating cells with native apyrase requires the enzyme to be active. Treatment of cell cultures with ADP or AMP, products of the reaction catalysed by apyrase, did not result in any change in cell viability (FIG. 2C). Since apyrase is cell-impermeative and, therefore, a mechanism to selectively destroy extracellular ATP, these results demonstrate that deprivation of extracellular ATP triggers a cell death response.

### Example 3

### Removal of External ATP by Treatment of A. thaliana Cell Suspension Cultures with Hexokinase and Glucose Results in Cell Death

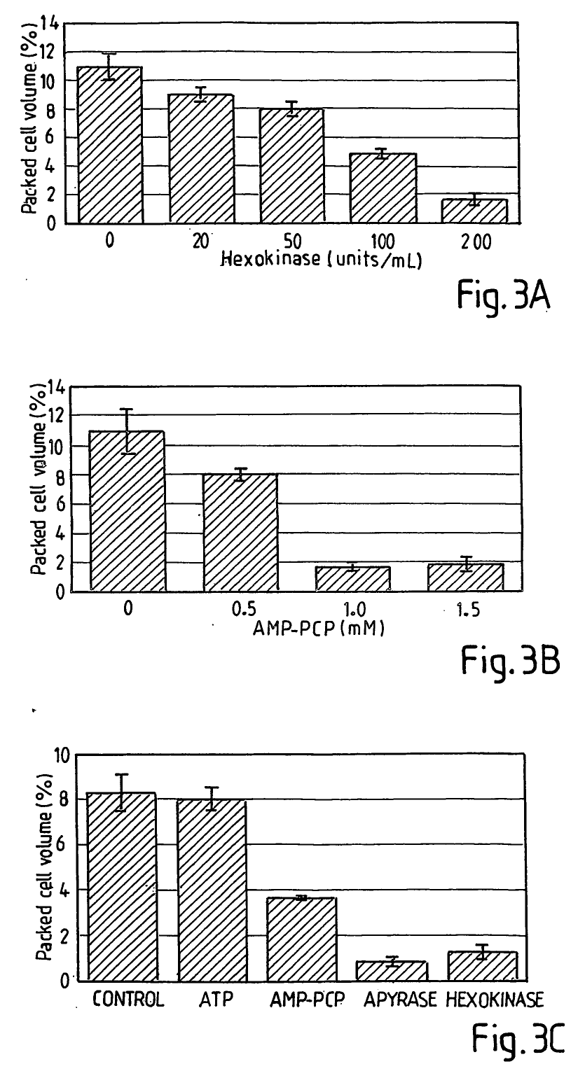

Arabidopsis cell cultures were grown and treated as described in example 2 and the glucose plus hexokinase treatment was used as the extracellular ATP removal system. The cultures were treated with a combination of 100 mM glucose and 0, 20, 50, 100, or 200 units/mL hexokinase. The results are shown in FIG. 3A. As apparent from the Figure, cell viability was progressively lost with increasing hexokinase concentration and 200 units/mL hexokinase and 100 mM glucose treatment was attended by an over 80% loss of viability. Although glucose can freely diffuse into cells, hexokinase is cell-impermeative and remains in the external medium, and its addition to cell cultures results in a targeted removal of extracellular ATP. Hexokinase that had been denatured by boiling for 5 minutes before addition to cell cultures did not cause cell death (FIG. 2B), demonstrating the absolute requirement for a native enzyme for cell death to ensue. Treatment of cells with ADP or glucose-6-phosphate, products of the reaction catalysed by hexokinase, did not affect cell viability (FIG. 2C). This result shows that removal of extracellular ATP compromises viability of Arabidopsis cells in suspension culture.

This finding was specific to plant cells: comparable experiments with combinations of glucose/hexokinase had no significant effect on the viability of the bacterium E. coli or the yeast S. cerevisiae (data omitted for brevity).

### Example 4

### Treatment of A. thaliana Cell Suspension Cultures with a Non-Hydrolysable ATP Analogue, AMP-PCP, Results in Cell Death

As cell death could be triggered by the removal of extracellular ATP, the inventors predicted that a compound that could compete with ATP metabolically or in signaling processes and is not hydrolysable, would also cause cell death. This was tested by the application of a non-hydrolysable analogue of ATP, β,γ-methyleneadenosine 5′-triphosphate (AMP-PCP), to Arabidopsis suspension cells. Because of its molecular size and charge, AMP-PCP cannot diffuse into cells and, therefore, selectively interferes with processes that utilise only extracellular ATP. The cell cultures were treated with 0, 0.5, 1.0, and 1.5 mM AMP-PCP in an experiment conducted as described in example 2. A 45.5 mM stock solution of AMP-PCP that had been adjusted to pH 6.5 using KOH was used. The dose-response of these cells to the ATP analogue is shown in FIG. 3B. Progressively increasing the concentration of AMP-PCP was accompanied by a loss in cell viability that reflected the occurrence of cell death due to a competitive exclusion of ATP from binding sites by the analogue. Treatment of cells with 1 mM ATP did not cause death of treated cells (FIG. 2C). These results confirm that extracellular ATP is required for the viability of suspension cells and that non-hydrolysable analogues of ATP can be used to kill cells.

### Example 5

### Treatment of Zea mays Suspension Cells with Apyrase, Hexokinase/Glucose, or a Non-Hydrolysable ATP Analogue Results in Cell Death

Black Mexican sweet corn cells were grown in MS medium with 2% (w/v) sucrose and 2 mg/L 2,4-dichlorophenoxyacetic acid, and adjusted to pH 5.7 with NaOH and HCl. The cultures were maintained by weekly sub-culturing 7 day old inoculum into fresh medium (10-fold dilution). Treatment of the cell cultures was performed 3 days after transferring to the fresh growth medium. A final concentration of 100 mM glucose was added to the culture before aliquoting 1.5 mL each into the plastic vials for treatment. The cell cultures were treated as described in example 2 using the following final concentrations; 1 mM ATP, 1 mM AMP-PCP, 100 units/mL apyrase, and 200 units/mL hexokinase. The control was treated with an equivalent volume of sterile deionised water alone. The response of corn cells to these treatments was similar to that of Arabidopsis cultures as shown in FIG. 3C. As expected, ATP did not affect the viability of the cells, but treatment with any of AMP-PCP, apyrase, or hexokinase/glucose caused significant cell death levels equivalent to those caused in the Arabidopsis cultures. This demonstrated that extracellular ATP is indispensable for the viability of a monocot (corn) cell system, as is the case in the dicot Arabidopsis.

### Example 6

### Exogenous Application of Extracellular Atp Depletion Systems to Areas of a Whole Plant Causes Cell Death and Development of Necrotic Lesions or Death of Entire Tissues

The effect of local application of extracellular ATP removal systems and a non-hydrolysable ATP analogue was evaluated on whole plants. Tobacco (Nicotiana tabacum), Arabidopsis, and bean (Phaseolus vulgaris) plants were sown in soil and raised in a growth cabinet with a 16-hour photoperiod at 20° C. and 8 hours of darkness at 15° C. The relative humidity was maintained at 60% and the photon flux density was 250 μmolm⁻² s⁻¹. The plants were used for treatment with the extracellular ATP removal systems when they were 5-6 weeks old. Only the cotyledons of bean plants were treated. The abaxial surface of a small zone of leaf tissue was treated by infiltrating the apoplast with the solution using a syringe and hypodermic needle. The three test solutions had the following concentrations of either: 0.5 units/μL apyrase, 1.85 units/μL hexokinase plus 100 mM glucose, or 1-5 mM AMP-PCP (pH 6.5). All three systems resulted in the development of necrotic lesions in the area where the application was made within 2 days of treatment (see, for example, FIG. 4A). Similar applications (control) without the active ingredients did not result in necrotic lesions.

In FIG. 4A, the arrow indicates the needle prick on the control leaf (top panel, (i)) or the localised necrotic lesions that develop after the application of one of the various extracellular depletion systems (ii=5 mM AMP-PCP; iii=apyrase; iv=hexokinase+glucose). Treatment with ATP (pH 6.5) or individual products from the reactions catalysed by apyrase and hexokinase did not result in cell death.

Similar results were obtained following treatment of tobacco leaves with: water (control, no necrosis); compared with the development of localised necrotic lesions after treatment with apyrase, or hexokinase plus glucose. Similarly, infiltration of 1 mM AMP-PCP caused localised tissue death whereas treatment with 1 mM ATP did not.

Again, comparable experiments with bean plants resulted in the development of necrotic lesions on leaves after treatment with apyrase or hexokinase and the lack of reaction of tissues treated with water or 100 mM glucose.

Localised treatment was used to show the contrast with adjacent living tissue. It is therefore clear that the cell death associated with the removal of extracellular ATP is not restricted to cells in suspension culture, but also applies to whole plants.

In order to demonstrate that the effect of extracellular ATP removal could be much more widespread than a localised necrotic lesion, the same depletion systems were applied to an entire leaf of the three plant species used above. The concentrations used were the same as defined above unless stated otherwise. This treatment resulted in the collapse of the treated tissues within 24 hours of treatment and then death of the entire leaf, as apparent in FIG. 4B (panels ii-iv), FIGS. 5A-C and 6A, B.

In FIG. 4B, panels ii-iv, the death of the entire leaf is apparent, whilst the control leaf (panel B(i)) remains healthy.

Similar results (shown in FIG. 5) were obtained with bean plants. FIG. 5A shows a healthy untreated control plant. FIG. 5B shows a plant with one control leaf treated with water (black arrowhead) and one leaf (white arrowhead) treated with apyrase. Likewise, a control leaf is denoted by a black arrowhead in FIG. 5C, whilst the white arrowhead indicates a leaf treated with hexokinase/glucose.

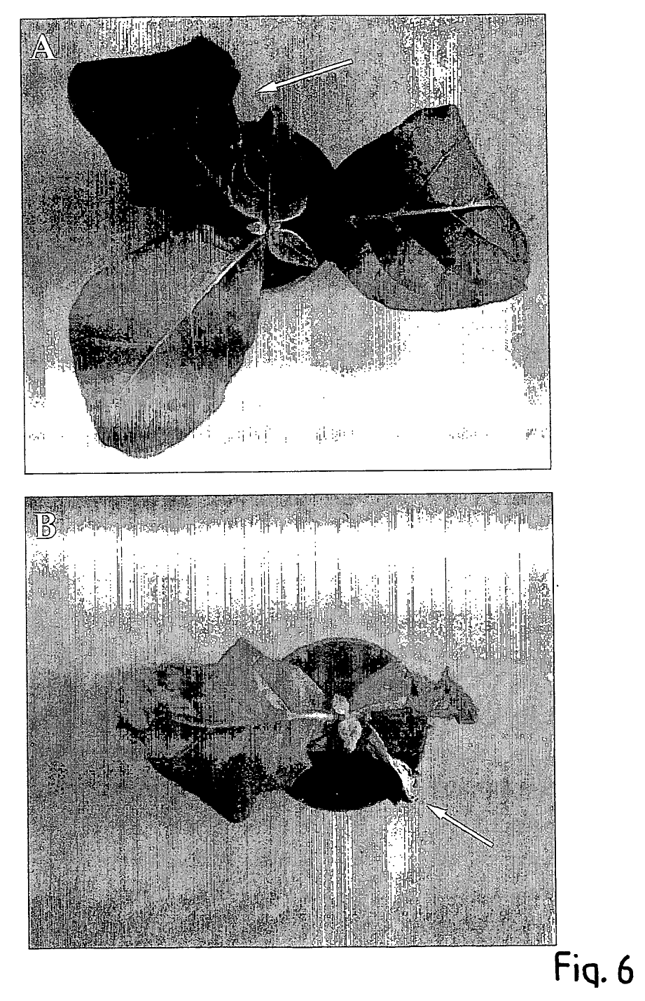

FIG. 6B shows a tobacco plant treated with 5 mM AMP-PCP. After 4 days, not only had the treated leaf died (white arrowhead), but so too had portions of some upper, untreated leaves, indicating that the AMP-PCP had become systemic. Comparable results were obtained following apyrase or hexokinase/glucose treatments (data omitted for brevity). The control plant (FIG. 6A) treated with an equivalent amount of ATP remained entirely healthy.

### Example 6B

Hydroponic Experiments

In order to further demonstrate that entire plants could be killed as a result of extracellular ATP depletion, experiments were conducted using hydroponic plant cultures.

Arabidopsis thaliana var. Columbia 24 were surface-sterilized as described before [Topping, J. F and Lindsey, K. (1991). “Shoot cultures and root cultures of tobacco”. In Lindsey, K (ed), Plant Tissue Culture Manual. Kluwer Academic Publishers, Dordrecht, the Netherlands, pp. A4: 1-13] and seeded onto 7 cm diameter, 1 cm thick polyurethane foam discs soaked in MS medium [0.22% (w/v) Murashige and Skoog salts, 1% (w/v) sucrose, adjusted to pH 5.7 with KOH/HCl]. The discs were placed in sterile phytatrays (Sigma) and incubated under a 16-hour light/8-hour dark cycle at 22° C. A week later, when the seeds had germinated and the roots had penetrated the foam, 30 ml of MS medium were added to the phytatray which caused the foam discs to float. The trays were transferred to a shaking platform (25 rpm.) under the same environmental conditions. By the second week, the roots of the plants had emerged through the other side of the foam and were in contact with the nutrient rich medium.

Two weeks after sowing the seeds, duplicate 500 μL aliquots were withdrawn from the growth medium of one such hydroponic culture of Arabidopsis over a 6-hour period. The amount of ATP in these samples was analysed by luminometry using the luciferase-luciferin method as described in Example 1. This analysis showed the presence of ATP released from the Arabidopsis plant roots and the increase in the amount of the secreted ATP over the 6 hour period of the experiment. The presence of ATP in the growth medium is consistent with our prediction that extracellular ATP secreted by the roots would accumulate in the medium since roots, unlike leaves, do not have a cuticle that retains exudates crossing the plasma membrane. The inventors appreciated that this experimental system could be useful for investigating the effects of extracellular ATP depletion in intact plants without invasive application methods of the depletion system as happens when needle-injection is employed.

Exactly 2 weeks from sowing, 4.2 mL of one of the following solutions were added (resulting in the indicated final concentration) to the growth medium of hydroponically grown Arabidopsis plants in a final volume of 50 mL made up using fresh growth medium:

Water

45 mM glucose

45 mM glucose+1 mM glucose-6-phosphate

45 mM glucose+5 mM glucose-6-phosphate

45 mM glucose+100 units/mL hexokinase

Three days after treatment, roots of the plants treated with glucose-hexokinase had started to turn brown and this browning intensified with time. The leaves of these plants became chlorotic within 4 days of treatment and they eventually died. Some of these leaves developed localised tissue death appearing in the form of brown lesions. Within 7 days of treatment, roots of these plants appeared completely dead and some of the shoots were dead or chlorotic and dying. Just like the water-treated controls, the roots of glucose and glucose+glucose-6-phosphate treated plants were the normal whitish cream colour and they had no dead leaves. Thus, glucose alone or glucose in combination with glucose-6-phosphate was not the cause of the death observed in the glucose-hexokinase treatments. This indicates that death caused by this treatment was due to depletion of ATP and not a result of production of glucose-6-phosphate.

Some of the leaves from plants under ATP depletion treatments developed localised necrosis that appeared as brown lesions. Within the 3 weeks that these plants were allowed to grow, the roots of control plants as well as plants treated with glucose or glucose combined with glucose-6-phosphate had grown to fill the base of the rectangular phytatrays holding the plants. In contrast, it was clear that the brown roots of plants treated with glucose-hexokinase had stopped growing. Using confocal microscopy, the inventors examined roots of these plants after staining with fluorescein diacetate as described in Example 2. This technique revealed that, whereas roots treated with glucose alone or in combination with glucose-6-phosphate were emitting green fluorescence, glucose-hexokinase treated roots failed to convert fluorescein diacetate to the fluorescent form, indicating that they were dead. This confirmed that the brown roots of plants treated with the ATP depleting system were indeed dead.

Overall, these results demonstrate that extracellular ATP is required for the viability of Arabidopsis roots just as it is indispensable for viability of the leaves. In addition, this non-invasive application of the ATP depletion system authenticates results obtained in experiments employing needle delivery of the ATP sequestering systems.

### Example 6C

Cell death is an essential part of plant development that is regulated by a programmed genetic template which may affect single cells, particular cell layers, or entire organs (Fukuda, 1997 Plant Cell 9, 1147-1156; Groover et al, 1997 Protoplasma 196, 197-211; Buchanan-Wollaston, 1997 J. Exp. Bot. 48, 181-199).

The inventors decided to investigate whether pathogen-induced hypersensitive cell death (Lam et al, 2001 Nature 411, 848-853) is mediated via hydrolysis of extracellular ATP. Treatment of plant cell cultures with an avirulent pathogen or pathogen-derived elicitors induces the hypersensitive cell death response (Levine et al, 1994 Cell 79, 583-593). The inventors reasoned that if this hypersensitive response were mediated via a transitory or sustained removal of extracellular ATP, then it might be abrogated by addition of excess exogenous ATP concomitant with, or shortly after, treatment.

Fusarium moniliforme elicitor prepared as described before (Raventos et al, 1995 Plant Journal 7, 147-155) was used in treatments of Arabidopsis cell cultures at a final concentration of 100 μg/ml. Pseudomonas syringae pv. tomato DC3000 strain, possessing the avirulence gene avrRPm 1, was grown in standard Luria Bertani medium (Sambrook et al 1989 Molecular Cloning: A Laboratory Manual. Cold Spring Harbor Laboratory Press, New York) with 50 μg/ml kanamycin A. Overnight bacterial cultures were harvested (1000×g for 10 min) and resuspended in water and used to inoculate Arabidopsis cell cultures at a final bacterial density of 8.8×10⁷ cfu/ml. A concentration of 1 mM ATP was used to investigate the effect of ATP on elicitor- or bacteria-induced death in Arabidopsis cell cultures.

The MTT assay (Watts et al, 1989 Int. J. Radiat. Oncol. Biol. Phys. 16, 939-942) was used to obtain a quantitative measure of plant cell viability 24 hours after the various treatments of the Arabidopsis culture.

The results of these experiments are shown in FIGS. 7A, B. FIG. 7A is a graph of extracellular ATP concentration (expressed as a percentage of that present in control cultures without Ps. syringae) against time (in hours). The level of extracellular ATP was measured using the luciferase-luciferin method and the ENLITEN^{RTM} kit (Promega, Southampton, UK) according to the manufacturer's instructions. FIG. 7A shows a transient depletion of extracellular ATP peaking about 6 hours after inoculation of the Arabidopsis cultures with an avirulent strain of Ps. syringae pv. tomato.

FIG. 7B shows the results of experiments in which Arabidopsis cultures were treated with Ps. syringae or with Fusarium elicitor, alone or with added exogenous ATP. The error bars represent the standard deviation from the mean (n=3). The presence of either Ps. syringae or the Fusarium elicitor caused a significant reduction in cell viability, which could be substantially negated by the inclusion of exogenous ATP in the cultures at 1 mM.

However, rescuing of cells from death by ATP was not via an antioxidant mechanism since ATP failed to prevent H₂O₂ accumulation (data omitted for brevity). This demonstrates that exogenous ATP does not indiscriminately block elicitor effects, but specifically inhibits cell death. Overall, these results are consistent with the conclusion that the cell death pathway mediated by hydrolysis of extracellular ATP is employed in nature during activation of the pathogen-induced hypersensitive response.

The results allow a rationalisation of extracellular ATP, cell death, and the response of plant cells to pathogen elicitors. Elicitors clearly affect cell viability (Levine et al, Cell 79, 583-593) and have been reported to activate membrane- and cell wall-bound ATPase activity (Kiba et al, 1995 Plant Cell Physiol. 36, 809-817). In plants, extracellular ATP hydrolysis is performed by ecto-apyrases (Komoszynski & Wojtczak, 1996 Biochim. & Biophys. Acta 1310, 233-241; Handa & Guidotti, 1996 Biophys. Res. Commun. 218, 916-923) and cell wall-bound ATPases (Kivilaan et al, 1961 Plant Physiol. 36, 605-610; Shiraishi et al, 1991 Plant Cell Physiol. 32, 1067-1075).

### Example 7

### Depletion of Extracellular ATP Alters the State of Protein Phosphorylation

The ability of a non-hydrolysable analogue of ATP to mimic the effects of external ATP depletion systems given in examples 2-3 suggested that extracellular ATP hydrolysis is required for maintenance of cell viability. The inventors predicted that removal of external ATP would be attended by changes in the phosphorylation status of some cellular derived proteins. In order to examine this, 30 mL of Arabidopsis cell cultures containing freshly added 100 mM glucose were treated by adding a sealed dialysis tube containing either 6000 units of hexokinase or water. The dialysis membrane had a molecular weight cut-off between 6- and 8 kDa, which confined hexokinase within the dialysis bag. Six hours later, the culture medium was separated from the cells by filtration through 2 layers of Miracloth and clarified by a 15-minute centrifugation at 3000×g. The culture medium proteins were precipitated by incubating at −20° C. in 80% acetone for 12 hours. Centrifuging for 10 minutes at 10,000×g pelleted the protein precipitates. The pellets were washed 3 times with 80% acetone and resuspended in a urea buffer (9M urea, 2M thiourea, 4% CHAPS, 1% DTT, 1% IPG buffer 4-7).

Aliquots containing 100 μg of protein were loaded into 7 cm IPGphor 4-7 gel strips (Amersham Biosciences, Amersham, UK) by the in-gel rehydration technique (Berkelman & Stenstedt 1998 2-D electrophoresis: Using immobilised pH gradients—Principles and Methods. Amersham Pharmacia Biotech, Buckinghamshire, UK). The proteins were separated by 2-dimensional SDS-polyacrylamide gel electrophoresis using standard procedures (Chivasa et al., 2002 Electrophoresis 23, 175-1765). One set of gels was stained with coomassie and the other was blotted onto nitrocellulose membranes and probed with a phosphotyrosine-specific antibody (Amersham Biosciences) using standard procedures (e.g., Chivasa et al., cited above).

As the inventors had predicted, it was observed that treatment of cell cultures with glucose/hexokinase alters protein phosphorylation. The results are shown in FIG. 8. The top panels show coomassie brilliant blue-stained gel sections of protein samples from glucose-treated (A) and glucose/hexokinase-treated (B) cell cultures. Bottom panels are western blots of similar gel sections that were immunoprobed with anti-phosphotyrosine serum. Panel C is glucose-treated and D is glucose/hexokinase-treated. In FIG. 8 (A and B), arrow heads indicate three secreted proteins whose abundance increased slightly in response to treatment with the glucose/hexolinase system as revealed by Coomassie blue staining. Western blots (FIG. 8, C and D) showed that these proteins contained phosphorylated tyrosine residue(s) and that treatment with glucose/hexokinase resulted in their apparent dephosphorylation. Protein spot 1 is completely dephosphorylated while the signal from spots 2 and 3 is significantly diminished but not totally abolished (FIG. 8D). This could arise from the sequestration of extracellular ATP that becomes unavailable to ecto-kinases responsible for phosphorylating the proteins. If ecto-phosphatases and ecto-kinases maintain the balance of phosphorylation of these proteins, then depletion of extracellular ATP by treatment tips the balance in favour of dephosphorylation. Without wishing to be bound by any particular theory, it is possible that changes in protein phosphorylation such as these could be involved in signaling cascades that give rise to cell death. Such phosphorylation changes could be targets for development of new herbicides.

### SUMMARY

The present inventors have noted that ATP can be removed from the external medium of cultured cells of both monocot and dicot plant species either by enzymatic cleavage of the gamma and beta phosphate groups with apyrase or by transfer of the gamma phosphate to glucose by hexokinase. ATP removal by either system results in cell death.

It is clear that: removal of external ATP results in cell death; mechanisms that override external ATP binding to its target(s) also result in cell death; the level of external protein phosphorylation alters following removal of external ATP and this may also be linked to death; any pathway involved down stream of the “message” resulting in cell death from the above will also result in cell death.

Those skilled in the art could use this basic experimental system to identify changes that occur at the metabolite, mRNA and/or protein level in plants, following such treatments. The skill bases for pursuing such studies are already in place and have been demonstrated (e.g., Ficarro et al., 2002 Nature Biotechnology 20, 301-305; Ideker et al., 2001 Science 292, 929-934). Proteomic analyses using either metabolic labeling, 1- and 2-dimensional gel electrophoresis, and protein identification techniques such as peptide mass fingerprinting, amino acid sequencing and immunology will allow for the identification of such proteins. Similarly, techniques for mRNA abundance determination will allow for the identification of potential gene candidates. Such techniques are readily available and include DNA chip technology, PCR based techniques, differential expression and Northern blot analyses. High throughput metabolic technologies are available to investigate changes in plant metabolites and these could be used to identify a potential block in metabolic pathways that are the result of this special form of “cell death”. Once the components have been identified, confirmation of their utility can be realised by gene knockout or gene silencing technologies or by the use of the proteins identified to develop specific inhibitors that will result in cell death. In an analogous fashion, components in the signal transduction pathway could be identified by the development of genetic screens based on lack of responsiveness to the extracellular ATP deprivation death pathway. A detailed description of how the person skilled in the art could identify potential targets for novel herbicidal compositions, and to use this information in turn to screen for suitable potentially herbicidal agents, is set out below in Example 8.

### Example 8

The identification of components in the NTP/ATP-depletion pathway can be achieved by a number of new technologies based on nucleic acid and protein technologies. Once these candidates are identified the corresponding protein can be used as the basis of selecting compounds which will bind to it, either covalently or non-covalently, and developed into specific inhibitors. The essentiality of these proteins for life can be tested by a variety of technologies including antisense, RNAi and identification of appropriate gene-disrupted lines [e.g. lines which are perhaps T-DNA tagged]. The following are given by way of example but are not the only methods of obtaining the identity of the target proteins, mRNA, cDNA and genes, and any of these methods may of course be performed in parallel to obtain confirmatory data.

### Example 8A

### Use of Protein Technology to Identify Targets

The key to this approach is to identity components which change in quantity or some other discernible characteristic following NTP/ATP-depletion or rescue from it. Arabidopsis cells, in culture, are treated with appropriate NTP/ATP-depletion conditions and at set points in time following NTP/ATP removal changes in the protein profiles are monitored. A comparison is made between the protein profile of treated and control cells. The cells are harvested at various time points, up to 24 hrs following the treatment, and protein extracts obtained following cellular disruption. Disruption is achieved by use of a French Press as described in Chivasa et al [Electrophoresis 23:1754-1765 [2002]] or by other mechanisms, including the use of glass beads—which is equally effective.

The samples are separated into a number of fractions to lower the complexity of the proteins in the sample and achieve greater resolution. These fractions are (i) total cell homogenate, (ii) cell wall fraction, (iii) soluble supernatant protein fraction following centrifugation at 120,000×g for 60 min and (iv) the microsomal pellet fraction from (iii). These samples are analysed by both 1D SDS-PAGE and 2D-gel electrophoresis. Samples are stained with one of a number of dyes to vizualise the proteins, including comassie brilliant blue, silver and Sypro Ruby red. The samples are imaged following eletrophoresis and staining using a ProXpressProteomics Imaging System [Perkin Elmer Life Sciences], quantified using image analysis software and the bands and spots which show changes identified. Spots and bands are picked using the Genomic Solutions ProPic work station and digested to produce peptides using an automated Genomic Solutions Progest robot. Following digestion, and sample work up, as described in Maltman et al (Electrophoresis 23:626-639, 2002) the proteins are identified by MALDI-TOF peptide mass fingerprinting, using a PE-Biosystems Voyager-DE STR mass spectrometer and searching of data bases using the PE Biosystems PS1 software.

Alternatively or additionally peptide sequencing may be performed with a Q-Star triple Quad mass spectrometer and the amino acid sequence of the peptides used to determine the protein from which it originates. As an alternative to the post-gel staining described above the gels could be prestained with two different Cy dyes and the differences identified using 2D-DIGE technology as described by Tong et al (Proteomics 1:377-396, 2001) and Orange P (Amersham Life Sciences News 5:2000). Following this the differentially expressed spots can be identified and the proteins identified as described above. Alternative technologies could be used to look at differentially expressed proteins by means other than gel separation techniques, such as Isotope Coded Affinity Tag techniques described in Ideker et al (Science 292:929-933, 2001) and Gygi et al (Nature Biotechnology 17:994-996, 1999) and mass spectroscopic identification. As one example of the sort of techniques available to those skilled in the art, the inventors have performed experiments as described below.

Suspension cell cultures were treated with 3 different systems that either deplete extracellular ATP or interfere with its binding to receptors or enzymes that utilize it. The 3 systems used were the nonhydrolysable ATP analogue AMP-PCP, the Fusarium elicitor, and the glucose-hexokinase system. In addition, the inventors expected that some, but not all, of the protein responses to the elicitors would be blocked or partially reversed by ATP. Thus, in the Fusarium elicitor system, the inventors included ATP only and a combination of ATP plus elicitor treatments. Another important aspect of this design was that, since ATP enhances cell growth, the response of proteins to ATP alone would be used to distinguish treatment-imposed changes from treatment-induced changes in protein profiles after ATP depletion.

For each system, at least 5 independent experiments were performed using cell suspension cultures generated independently. These constituted 5 biological replicates and ensured the exclusion of non-reproducible events. Although the 3 systems activate the same pathway, differences in the way the primary stimulus (viz. permanent enzymatic extracellular ATP depletion, transient elicitor-induced extracellular ATP depletion, and inundation with nonhydrolysable ATP) is applied could inherently result in the identification of equally important common as well as unique protein targets. Thus, proteins that respond to a minimum of 2 of the systems were taken as potential candidates with a role in the cell death pathway.

It is clear that this approach can be used as a filter to enable identification of proteins with a potential signaling role in the novel death pathway. However, the definitive proof of function for the proteins identified using this methodology will come from approaches such as the one described in 8B below. This method is applicable to the analysis of proteins from various cell fractions and has been used in the current work to analyse the soluble protein fraction. Therefore, any person skilled in the art can use the same approach to identify proteins from other cellular fractions e.g., the microsomal and organellar fractions.

### Example 8B

Experimental Methods

B(i). Fusarium Elicitor

For experiments using the Fusarium elicitor, 6 independent experiments were performed. Arabidopsis cell suspension cultures were grown in the dark for 3 days and 4.75 mL aliquots were exposed to the following treatments in a final volume of 5 mL by mixing with 250 μL of the solutions below;
2. Control—water [250 μL],
3. ATP—a final concentration of 1 mM ATP [50 μL of a 100 mM pH 6.7 stock plus 200 μL water],
4. F400—a final concentration of 400 μg/mL Fusarium elicitor [200 μL of a 10 mg/mL elicitor stock plus 50 μL water], and
5. AF-400—a combination of both 1 mM ATP and 400 μg/mL Fusarium elicitor [200 μL of 10 mg/mL elicitor plus 50 μL of 100 mM ATP pH 6.7].

After 24 hours of incubation with the treatment solutions, the cells were harvested by filtering through 2 layers of Miracloth and resuspended in 300 μL of 10% trichloroacetic acid in 1.5 mL microfuge tubes. The acidified cells were snap-frozen in liquid nitrogen and stored at −20° C. The cells were thawed and homogenised using plastic micropestles in the presence of 100 mg of sand. The homogenates were centrifuged for 10 minutes at 16,000×g. The supernatants were discarded and the pellets washed 3 times with 500 μL 80% acetone by repeated resuspension and centrifugation. The final pellets were washed once with the same volume of absolute acetone and dried by briefly blowing compressed air over them. Precipitated protein was extracted by resuspending the pellets in 500 μL of a urea buffer (9M urea, 2M thiourea, 4% CHAPS) and incubating at room temperature on an orbital shaker (180 r.p.m) for at least 30 minutes. Insoluble material was removed by centrifuging for 10 minutes at 16,000×g and discarding the pellets.

Sample Clean-Up and Fluorescent Labeling

Protein aliquots of 100 μL each from these preparations were stripped of non-protein contaminants using an Amersham Biosciences 2-D Clean-Up Kit following the manufacturer's instructions. The cleaned protein was resolubilised in a Tris-buffered solution (9M urea, 2M thiourea, 4% CHAPS, 30 mM Tris-Cl pH 9) and adjusted to pH 8.5 using NaOH. Protein concentration was determined by a modified Bradford assay (Ramagli & Rodriguez 1985 Electrophoresis 6, 559-563) against a bovine serum albumin standard.

Stock solutions of CyDye DIGE Fluor (Amersham Biosciences, UK) were prepared by reconstituting the lyophilised powder in dimethylformamide (DMF) to a final concentration of 1 nmole/μL. The stock was further diluted 2 in 5 using DMF to give a working solution of 400 pmol/μL. Sample aliquots containing 50 μg protein in 18 μL buffer (9M urea, 2M thiourea, 4% CHAPS, 30 mM Tris-Cl pH 8.5) were spiked with 1 μL of the CyDye working solution and incubated for 30 minutes on ice in the dark. The labeling reaction was terminated by the addition of 1 μL 10 mM lysine. For labeling protein amounts over 50 μg, the volumes of all solutions were scaled up proportionally to maintain the same ratio of 400 pmol CyDye to 50 μg protein in 20 μL final reaction volume. Control as well as Fusarium elicitor-treated samples were labeled with CyDye DIGE Fluor Cy3, while ATP-treated and ATP/Fusarium-treated samples were labeled with CyDye DIGE Fluor Cy5. A pooled standard containing 25 μg protein from each sample was labeled with CyDye DIGE Fluor Cy2.

2-D Electrophoresis and Image Acquisition

Six independent experiments were conducted and the total number of protein samples was 24. However, protein labeling and gel analysis was performed on 3 experiments at a time and then all the six experiments were combined during analysis of biological variation using the Amersham Biosciences DeCyder BVA software.

Aliquots with 12.5 μg labeled protein each were mixed as shown in Table 1. The volume of the mixtures were made up to 70 μL to give a final concentration of 9M urea, 2M thiourea, 4% (w/v) CHAPS, 1% (w/v) DTT and 2% IPG buffer pH 4-7. Immobiline DryStrips (18 cm pH 4-7 linear, Amersham Biosciences) were rehydrated overnight using sample buffer (9M urea, 2M thiourea, 4% CHAPS, 1% DTT, 2% IPG buffer pH 4-7). The protein mixtures were loaded in to the rehydrated first dimension gels using the cup-loading technique. Isoelectric focussing (IEF) was performed using the Ettan IPGphor (Amersham Biosciences). During IEF, the temperature was kept at 25° C. and a maximum current of 50 μA per gel was set. A total focussing of 70 kVh was achieved by following a running protocol with 4 phases of stepped voltages from 500 to 6,500V. Prior to second dimension, the gels were equilibrated, reduced and alkylated as described previously (Chivasa et al., 2002 Electrophoresis, cited previously). The proteins were separated in 12% polyacrylamide second dimension gels using the Ettan DALTtwelve System (Amersham Biosciences). These analytical gels were initially run at 5 W/gel for 30 minutes and subsequently at 17 W/gel until the bromophenol blue reached the bottom of the gels.

TABLE 1
2-D DIGE experimental design for biological variation analysis

| Gel No. | Cy2 | Cy3 | Cy5 |
|---|---|---|---|
| 1 | Pooled standard | Control expt. 1^{a} | ATP expt. 1 |
| 2 | Pooled standard | Control expt. 2 | ATP expt. 2 |
| 3 | Pooled standard | Control expt. 3 | ATP expt. 3 |
| 4 | Pooled standard | F-400 expt. 1 | AF-400 expt. 1 |
| 5 | Pooled standard | F-400 expt. 2 | AF-400 expt. 2 |
| 6 | Pooled standard | F-400 expt. 3 | AF-400 expt. 3 |

^{a}expt. 1 denotes experiment number 1; F-400 denotes Fusarium elicitor treatment; AF-400 denotes a treatment with both Fusarium elicitor and ATP

Gel images were acquired by scanning the gels with the Typhoon 9400 variable mode imager (Amersham Biosciences). Cy2 images were scanned using a blue laser (488 nm) at an emission wavelength of 520/40 nm (maxima/band width). A green laser (532 nm) was used to scan Cy3 images at an emission wavelength of 580/30 nm. Cy5 images were acquired after excitation with a red laser (633 nm) using an emission filter of 670/30 nm. All gels were scanned at a resolution of 100 μm using PMT voltages that did not allow saturation of the most intense spot on each image. The DeCyder™ Differential Analysis Software Version 4.00 (Amersham Biosciences, UK) was used for gel analysis.

B (ii). Nonhydrolysable ATP Analogue

Aliquots of 5 mL each of Arabidopsis cell suspension cultures grown in the dark for 3 days were treated with water (serving as controls) or 50 μM AMP-PCP and harvested for protein extraction 72 hours later. Protein extraction and processing for DIGE labeling were performed as described above. All control samples were labeled with CyDye DIGE Fluor Cy3, while AMP-PCP treated samples were labeled with CyDye DIGE Fluor Cy5. A pooled standard containing 25 μg protein from each sample was labeled with CyDye DIGE Fluor Cy2. Aliquots with 12.5 μg labeled protein each were mixed as shown in Table 2 and separated by 2-DE and imaged as described above.

TABLE 2
2-D DIGE experimental design for biological variation analysis

| Gel No. | Cy2 | Cy3 | Cy5 |
|---|---|---|---|
| 1 | Pooled standard | Control expt. 1^{a} | AMP-PCP expt. 1 |
| 2 | Pooled standard | Control expt. 2 | AMP-PCP expt. 2 |
| 3 | Pooled standard | Control expt. 3 | AMP-PCP expt. 3 |
| 4 | Pooled standard | Control expt. 4 | AMP-PCP expt. 4 |
| 5 | Pooled standard | Control expt. 5 | AMP-PCP expt. 5 |

^{a}expt. 1 denotes experiment number 1

B (iii). Glucose-Hexokinase

Aliquots of 5 mL each of Arabidopsis cell suspension cultures grown in the dark for 3 days were treated with 100 mM glucose (serving as controls) or a combination of 100 mM glucose plus 200 units/mL hexokinase (Hk). The cells were harvested 48 hours later and protein extracted and processed via 2-D DIGE as described for AMP-PCP above.

Preparative Gels, Mass Spectrometry and Protein Identification

Aliquots with 200 μg and 400 μg of unlabeled protein from all the treatments were separated 2-D electrophoresis as described above. These preparative gels were fixed in a solution containing 40% (v/v) methanol and 10% (v/v) glacial acetic acid. After 12 hours, the gels were incubated with fresh fixing solution for another 12 hours and then gels with 200 μg protein were stained overnight with Sypro™ Ruby solution in the dark (Genomic Solutions, Huntingdon, UK). These gels were destained for 4 hours by incubating with 10% (v/v) methanol 6% (v/v) acetic acid. Imaging was performed using the Typhoon 9400 with at an excitation wavelength of 532 nm and 610/30 nm emission filter. The images were matched back to DIGE analytical gels using DeCyder software and a picking list of proteins of interest was generated. The same gels were re-imaged on a ProPick Workstation (Genomic Solutions) and the protein spots of interest excised from the gels for processing by mass spectrometry.

The gels with 400 μg protein were stained with silver as follows. After fixing, the gels were incubated for 30 minutes in sensitizing solution [30% (v/v) methanol; 6.8% (w/v) sodium acetate; 0.2% (w/v) sodium thiosulphate] and washed with water 3 times by incubating for 10 minutes each wash. After incubation for 40 minutes in 0.25% (w/v) silver nitrate, the stain was developed until the protein spots were visible against a clear background using a solution containing 2.5% (w/v) sodium carbonate and 0.04% (v/v) formaldehyde. The reaction was stopped with 1.46% (w/v) EDTA. Highly abundant protein spots of interest were excised from the Sypro™ Ruby stained gels while medium to low abundant proteins were picked from the silver stained gels. Proteins were identified by MALDI-Tof as described previously (Simon et al., 2002 Proteomics 2, 1735-1742). The peptide masses generated via MALDI-ToF were used to search the NCBI database (National Center for Biotechnology Information), found at http://www.ncbi.nlm.nih.gov. and the Mowse score cut off point for a positive identification was set at 64.

8C. Identification of Putative Candidates of the Cell Death Pathway

The inventors identified a total of 72 protein spots that corresponded to 59 distinct gene products proteins) in the NCBI database. However, when the sequences were used to search the MIPS database (http://mips.gsf.de/proj/thal/db/index.html) using the BLAST search engine on the same site, a total of 57 distinct gene products were identified. The list of the proteins and their accession numbers is given in Table 3 and appendix-1 shows the details of the responses of each protein spot to the different treatments.

TABLE 3
List of proteins that are potential components of the cell
death pathway activated by extracellular ATP depletion.

| Num- | MIPS | NCBI | |
|---|---|---|---|
| ber | accession # | accession # | Protein name |
| 1 | At2g04030 | H84453 | Probable heat shock protein |
| | | | [imported] |
| 2 | At3g09440 | gi15232682 | Heat shock protein hsc70-3 |
| 3 | At5g02490 | gi15241847 | Heat shock cognate 70 kDa protein |
| | | | 2 (hsc70-2) |
| 4 | At5g02500 | gi1072473 | dnaK-type molecular chaperone |
| | | | hsc70.1 |
| 5 | At1g78900 | gi15219234 | Vacuolar ATP synthase catalytic |
| | | | subunit A |
| 6 | At4g38510 | gi2493132 | Vacuolar ATP synthase subunit B |
| | | | isoform 2 |
| 7 | At5g08690 | gi18415911 | H+-transporting ATP synthase |
| | | | beta chain |
| 8 | Atp1 | gi14916970 | ATP synthase alpha chain, |
| | | | mitochondrial |
| 9 | At5g28540 | gi15241844 | Luminal binding protein 1 |
| | | | precursor (BiP-1) |
| 10 | At5g42020 | gi30693966 | Luminal binding protein 2 (BiP-2) |
| 11 | At2g30860 | gi15224581 | Putative glutathione S-transferase |
| 12 | At2g30870 | gi15224582 | Putative glutathione S-transferase |
| 13 | At1g02920 | gi15218639 | Putative glutathione S-transferase |
| 14 | At1g02930 | gi15218640 | Putative glutathione S-transferase |
| 15 | At4g02520 | gi2554769 | Chain A, Structure Of glutathione |
| | | | S-transferase |
| 16 | At4g11600 | gi20138157 | Phospholipid hydroperoxide |
| | | | glutathione peroxidase |
| 17 | At1g07890 | gi15223049 | L-ascorbate peroxidase 1, |
| | | | cytosolic (APX1) |
| 18 | At3g17390 | gi15229033 | Putative S-adenosylmethionine |
| | | | synthetase |
| 19 | At4g01850 | gi15234354 | S-adenosylmethionine synthetase 2 |
| 20 | At4g13940 | gi15236376 | Adenosylhomocysteinase |
| 21 | At2g36530 | gi15227987 | Enolase (2-phospho-D-glycerate |
| | | | hydrolase) |
| 22 | At3g04120 | gi3435286 | Glyceraldehyde-3-phosphate |
| | | | dehydrogenase C subunit (GapC) |
| 23 | At3g04120 | gi21593240 | Glyceraldehyde-3-phosphate |
| | | | dehydrogenase C subunit (GapC) |
| 24 | At2g29550 | gi15227559 | Tubulin beta-7 chain |
| 25 | At5g19770 | gi15241168 | Tubulin alpha-3/alpha-5 chain |
| | At5g19780 | | (TUA3) |
| 26 | At3g48000 | gi15228319 | Putative mitochondrial aldehyde |
| | | | dehydrogenase |
| 27 | At1g72330 | gi21954069 | Putative alanine aminotransferase |
| 28 | At1g17290 | gi21954071 | Putative alanine aminotransferase |
| 29 | At1g17290 | gi9082270 | Alanine aminotransferase |
| 30 | At3g10050 | gi15232827 | Threonine ammonia lyase |
| 31 | At3g17820 | gi15229530 | Glutamine synthetase-related |
| | | | protein |
| 32 | At3g53180 | gi11358581 | nodulin/glutamate-ammonia ligase- |
| | | | like protein |
| 33 | At1g53240 | Gi18404382 | Malate dehydrogenase [NAD], |
| | | | mitochondrial |
| 34 | At3g15020 | gi15232468 | Malate dehydrogenase [NAD], |
| | | | mitochondrial |
| 35 | At3g47520 | gi25283601 | Malate dehydrogenase [EC 1.1.1.37], |
| | | | chloroplast |
| 36 | At5g37510 | gi30693102 | NADH dehydrogenase (ubiquinone), |
| | | | mitochondrial |
| 37 | At2g21330 | gi14334740 | Putative fructose-bisphosphate |
| | | | aldolase |
| 38 | At2g01140 | gi15226185 | Putative fructose-bisphosphate |
| | | | aldolase |
| 39 | At1g09130 | gi18390982 | ATP-dependent Clp protease |
| | | | proteolytic subunit |
| 40 | At5g35590 | gi20260140 | Multicatalytic endopeptidase |
| | | | complex alpha subunit |
| 41 | At3g22630 | gi15228805 | 20S proteasome beta subunit D |
| | | | (PBD1) |
| 42 | At3g58610 | gi288063 | Ketol-acid reductoisomerase |
| 43 | At5g42740 | gi11094242 | Cytosolic phosphoglucose isomerase |
| 44 | At3g17240 | gi30684419 | Dihydrolipoamide dehydrogenase 2, |
| | | | mitochondrial |
| 45 | At1g76680 | gi21593388 | 12-oxophytodienoate reductase |
| | | | (OPR1) |
| 46 | At5g16970 | gi15237888 | Putative NADP-dependent |
| | | | oxidoreductase (P1) |
| 47 | At1g18450 | gi21593375 | Unknown protein |
| 48 | At4g34350 | gi7485313 | Hypothetical protein F10M10.120 |
| 49 | At3g62530 | gi18412406 | PBS lyase heat-like repeat- |
| | | | containing protein |
| 50 | At2g17280 | gi15227803 | Phosphoglycerate/ |
| | | | bisphosphoglycerate mutase family |
| | | | protein |
| 51 | At3g22850 | gi15228883 | Expressed protein |
| 52 | At2g41530 | gi21593226 | Putative esterase D |
| 53 | At5g59880 | gi30697295 | Actin depolymerizing factor 3- |
| | | | like prptein |
| 54 | At1g15930 | gi15218373 | 40S ribosomal protein S12 (RPS12A) |
| 55 | At1g21750 | gi30687521 | Putative protein disulfide |
| | | | isomerase |
| 56 | At4g04950 | gi15234516 | Putative thioredoxin |
| 57 | At5g54500 | gi15239652 | Putative quinone reductase |
| 58 | At3g13920 | gi14594802 | Translation initiation factor |
| | | | elf-4A1 |
| 59 | At1g79550 | gi21536853 | Putative phosphoglycerate kinase |

These proteins were selected based on the following criteria:

(1) The change in abundance in response to the treatment had to be significant at the 95% confidence level.

(2) The same protein spot had to respond to at least 2 ATP-depleting treatments or satisfy either 3 or 4 below.

(3) If a protein spot responded to only 1 treatment, then a neighbouring spot with the same gene identity or an isoform of the same protein should respond to a different treatment(s). Thus, even though the 2 treatments are not activating the same protein spot, they do converge on the same protein family, indicating that the protein family could be vitally important in this pathway. Or,

(4) The response of the spot to ATP treatment should be in the opposite direction from its response to the “ATP-depleting” system e.g., if a spot increased in abundance to AMP-PCP treatment only, it would be selected if it decreased in abundance in response to ATP treatment.

### Example 8D

Protein fractions could be prepared in the same way as in Example 8A above but in order to identify new proteins which were synthesised in response to extracellular ATP depletion (or rescue therefrom) the cells would be labelled with 35S methionine or other radioactive amino acids. In this way following sample fractionation, as above, and fluorography following electrophoresis, candidate proteins which were newly synthesised and differed between control and test samples can be identified. This has the advantage that new proteins which are synthesized in response to the altered conditions of the cell can be selectively identified within a background of other cellular proteins.

### Example 8E

Arabidopsis cell suspensions are treated with water [sample a] or Fusarium elicitor [sample b] and a third reaction is performed using the same elicitor but in the presence of 1 mM ATP [sample c]. The three samples are compared using the variety of techniques described in Examples 8A and 8B, revealing specific changes attributable to activation of the elicitor mediated cell death pathway which could be reversed by addition of ATP.

### Example 8F

### Use of Nucleic Acid Technology to Identify Candidates in the Cell Death Pathway

Experiments are performed using ATP-depletion with controls as described in examples 8A-E above. At set time points following treatment, from 5 min to 2 hrs, mRNA is extracted from each of the samples. RNA may be extracted from Arabidopsis cell suspension cultures using a modification of a published method (Fuerst et al 1996 Plant Physiol. 112, 1023-1033). Essentially, cells are harvested by vacuum filtration through cellulose filters, flash-frozen in liquid nitrogen and RNA is extracted using the QIAGEN RNeasy kit, followed by washing in 3M sodium acetate to remove contaminants. Fifty microgrammes of total RNA are used to make cDNA by reverse transcription using standard protocols (e.g. Sambrook et al, Molecular Cloning. A laboratory Manual. Second edition, Cold Spring Harbor Laboratory Press) from each of the samples in a particular treatment. cDNA samples will be analysed for transcriptional profiles using the Affymetrix oligonucleotide GeneChip (Harner et al, 2000 Science 290, 2110-2113), which contains representation of ca. 24,000 Arabidopsis genes. Double-stranded cDNA is transcribed to form biotin-labelled cRNA, which is fragmented by metal hydrolysis prior to hybridization to the GeneChip. Genes which are differentially expressed over 2-fold are identified following cluster analyses. The alteration in expression of these genes is confirmed using both real-time PCR and Northern analyses. RT-PCR and northern analysis on Arabidopsis RNA samples may be carried out using published protocols (e.g. Casson et al, 2002 Plant Cell 14, 1705-1721). In this way genes, the expression of which is altered following ATP depletion, could be identified and, in particular, early and late responding genes could be identified.

### Example 8G

### Use of Fungal Elicitors

Experiments are performed using Fusarium elicitor, with and without 1 mM ATP addition, using Arabidopsis suspension cultures as described in Example 8C above. At set time points following treatment, from 5 min to 2 hrs, mRNA is extracted from each of the samples. Genes with an altered expression in response to Fusarium elicitor treatment, which alteration is reversible by ATP addition, may be identified using protocols described above in Example 8D. These genes represent potential targets for new herbicides.

### Example 8H

### Development of Screens for Potential Herbicides

One type of screening procedure might involve a number of steps:
1. Verification that the target gene is essential to life. This may be done, for example, by means of RNAi; looking for T-DNA tagged lines, in which the gene is disrupted and which are lethal in the homozygous state; and/or antisense technology.
2. Over-express the proteins and look for chemicals which will bind to them and inhibit activity—mass screen.
3. Transform and express the plant protein in a microbe, having deleted the corresponding (functionally homologous) gene from the organism—(so that it is dependent on the plant protein for growth) then conduct a differential microbial screen using wild type vs transformed strains with a variety of chemical compounds.

An alternative screening approach might be based on the realisation by the inventors that inhibitors of ecto-ATPases and ecto-phosphatases, such as ATP-γ-S, can have herbicidal effects in their own right (i.e. be active agents, rather than merely potentiate toxic effects of xenobiotics).

Thus, screening for compounds which are active as inhibitors of plant ectophosphatases should identify lead compounds with herbicidal activity. Such a screening method, with high throughput, is disclosed as Example 6 in WO 01/64859 (the content of which is incorporated herein by reference). WO 01/64859 also discloses a number of stable compounds (having structural formulae I-XIX) which were identified by such a screen and may thus have activity as herbicidal compounds in their own right, independently of potentiating the effects of exogenous xenobiotics.

### Example 9

### Herbicidal Compounds

The inventors tested a range of compounds for their potential herbicidal activity. These consisted of several nonhydrolysable ATP analogues as well as nonhydrolysable analogues of GTP, another nucleotide triphosphate. Arabidopsis plants were grown hydroponically for 2 weeks as described in Example 6B. These plants, still attached to the polyurethane foam discs, were then transferred to phytatrays that contained sand. Plants grown under high humidity conditions, achieved in these enclosed phytatrays, tend to have poor or no development of the waxy cuticle and so are ideal for spray application of solutions with the aid of common surfactants like Tween 20. The plants in each tray were sprayed with 2 mL of an aqueous solution adjusted to pH 6.7 with KOH and containing 5 mM active ingredient and 0.02% (v/v) Tween 20. Control plants were sprayed with the same solution containing only Tween 20 but no active ingredient.

Within 3 days of spraying, the plants treated with the phytotoxic compounds as indicated in Table 4 had collapsed and exhibited advanced death of the tissues. Both the nonhydrolysable ATP analogues, AMP-PNP and ATP-γ-S, had herbicidal activity. Treatments with equimolar concentrations of the lithium salt of ADP were not phytotoxic, proving that the Li⁺ ions were not the cause of the toxicity observed with the lithium salt formulations of the nonhydrolysable analogues. Thus, the ATP moiety was the component in the solutions that caused death. Nonetheless, some reports claim that ATP-γ-S is slowly hydrolysed and may cause irreversible thiophosphorylation of proteins. Because of this possibility, we included an equimolar sodium thiophosphate (NaPO₃S) control which did not cause death, demonstrating that only intact ATP-γ-S is herbicidal, but not the breakdown products. Since the ATP-treated plants remained healthy, the lack of hydrolysis of the bond between the beta and gamma phosphates in the analogues appeared to be the cause for their potency.

The inventors also found that GTP-γ-S, a nonhydrolysable analogue of GTP, has herbicidal properties while GTP does not (Table 4). All the phytotoxic compounds were able to kill the plants, whether they had a waxy cuticle or not.

TABLE 4
Effects of different compounds on Arabidopsis
plants after foliar application

| Compound | Formulation | Effects |
|---|---|---|
| Tween 20 | Pure liquid | None |
| ADP | Lithium salt | None |
| ATP | Sodium salt | None |
| GTP | Sodium salt | None |
| NaPO₃S | Sodium salt | None |
| AMP-PNP | Lithium salt | Phytotoxic |
| ATP-γ-S | Lithium salt | Phytotoxic |
| GTP-γ-S | Lithium salt | Phytotoxic |

The inventors also examined the potential herbicidal effects of AMP-PCP, another nonhydrolysable ATP compound, on 3 weeks soil-grown tobacco plants in 9 cm diameter plastic petri-dishes. The plants were treated with a solution of 0.02% (v/v) Tween 20 as a control, 5 mM ATP in 0.02% Tween 20, or 5 mM AMP-PCP in 0.02% Tween 20. Plants in each petri-dish were sprayed with 2 mL of the treatment solution. Three days after treatment, plants treated with AMP-PCP started to die while those treated with ATP and the controls were unaffected. All the plants in the AMP-PCP treated dishes eventually died and dried up. These results show that use of the nonhydrolysable ATP analogues as potential herbicides is not limited to Arabidopsis, but can be extended to other plants as indicated by the reactions of tobacco plants to AMP-PCP.

### Example 10

Functional Analysis of Proteins Identified as Candidate Signaling Pathway Components

2D-DIGE has led to the identification of Arabidopsis proteins that are differentially expressed in response to conditions that either induce or suppress the ATP-mediated cell death (AMCD) pathway. To further characterize the function of these proteins, two approaches will be used: first, the identification and phenotypic analysis of mutants defective for the genes encoding the identified proteins; and second, the use of RNA interference (RNAi) to down-regulate the levels of mRNA, and so the protein levels, encoded by the genes of interest. The objective is to determine whether the proteins/genes identified are essential for either cell viability or cellular responses to ATP depletion.

Mutant Analysis

Internationally there are available several collections of insertional mutants, generated by T-DNA insertional mutagenesis, which can be screened for mutations in genes of interest. Public access to these collections is possible through the world-wide web, and mutant seed can be ordered through the Arabidopsis stock centres, such as the Nottingham Arabidopsis Stock Centre (NASC, School of Biosciences, University of Nottingham, Loughborough LE12 5RD). Once seed is obtained, plants are grown up individually and genotyped, using PCR amplification of insertion event-specific genomic sequences, mediated by oligonucleotide primer pairs specific for the T-DNA and the gene of interest. The design of the primer sequences is determined by the identity of the gene of interest and the sequence of the T-DNA insertion element used to generate the mutation. The purpose of the genotyping is a) to confirm that the T-DNA is in the gene of interest, and b) to determine whether individual plants are homozygous or heterozygous for the T-DNA mutation. Southern blot analysis will be carried out to determine the T-DNA copy numbers in individual transgenic lines. Genomic DNA will be isolated and digested by restriction enzymes (the precise enzymes used depending on the T-DNA used for mutagenesis), size fractionated, blotted onto nylon membranes and probed with a radio-labelled or chemiluminescent probe, derived from T-DNA sequence, according to standard methods (e.g. as described by Wei et al. 1997, Plant Journal 11, 1307-1314). Restriction enzymes that cut both within the T-DNA and in flanking genomic DNA are used, to allow a precise determination of T-DNA copy number. Outcrossing mutants to wild-type plants, in order to generate single copy insertion lines by genetic segregation, will be carried out where necessary, again according to standard methods for crossing Arabidopsis (e.g. Topping et al. 1997, Development 124, 4415-4424).

Plants heterozygous for single copy T-DNAs will be allowed to self-fertilize, and seed will be collected, for genetic analysis to characterize the segregation of any mutant phenotypes. This seed will be surface-sterilized, vernalized and germinated in vitro on hormone-free medium, for preliminary phenotypic analysis, according to standard methods (e.g. Souter et al. 2002, Plant Cell 14, 1017-1031). If germination is found to fail in at least about 25% of the seeds, this is indicative of an embryonic-lethal phenotype associated with a homozygous recessive mutation. This would be preliminary evidence that the mutant gene is required for cell viability or function during embryogenesis (and possibly at other stages of the plant life-cycle). If all, or nearly all (at least 90%) seeds germinate, and at least about 25% of seedlings show a defective phenotype, this would be preliminary evidence that the mutant gene is required for cell viability or function during seedling development, and possibly also embryogenesis. If all, or nearly all, seeds germinate, and none of the seedlings show a defective phenotype, this could suggest at least three possibilities that would require further investigation: (i) it could represent evidence that the gene of interest is not involved in the AMCD pathway; (ii) it could suggest that the gene is involved in the pathway, but is functionally redundant with one or more functionally related genes; or (iii) it could suggest that a mutant phenotype is conditional on other factors not being tested under the growth conditions used.

To determine whether the latter case is relevant, ‘gauntlet screens’ will be carried out on putative AMCD pathway mutants. The rationale is that some mutants may show defective responses to only some experimental treatments, depending on where the protein acts in the AMCD pathway. Potentially this provides a means to dissect the pathway genetically. Screens will include the analysis (in any of the methods disclosed herein) of seedling response to treatment with: AMP-PCP, apyrase, glucose-hexokinase or fungal elicitor; and the cell death response will be monitored. To carry out the phenotypic response screens, seedlings of mutants will be grown hydroponically and challenged by the addition of chemical treatments to the roots. Cell death will be monitored as frequency of root necrosis.

Any mutants showing defective phenotypes or responses to treatments designed to induce cell death will be characterized further, by genetic complementation with the wild-type allele of the respective mutant gene. The rationale here is that, if the mutant gene is functionally related to the observed mutant phenotype, then a wild-type phenotype or response will be rescued by the expression of the wild-type allele. The wild-type gene will be introduced into the mutants under the transcriptional control of ca. 2 kb of its own promoter, and F1 seedlings will be characterized for phenotypic rescue. This will be carried out using methods standard for genetic complementation analysis of Arabidopsis mutants in this laboratory (e.g. Casson et al. 2002, Plant Cell 14, 1705-1721; Souter et al. 2002, Plant Cell 14, 1017-1031).

RNAi

If insertional mutants are not available for the genes of interest, down-regulation will be carried out using RNAi. The wild-type cDNA will be cloned into an RNAi vector developed in this laboratory and shown to be effective (Dean et al. 2004 Plant Molecular Biology, in press) and introduced into Arabidopsis by Agrobacterium-mediated transformation, using methods standard in this laboratory (e.g. Casson et al. 2002, Plant Cell 14, 1705-1721). Seeds of primary transformants (T1 plants) will be selected on the basis of resistance to kanamycin, conferred by the RNAi transformation vector. These plants will be grown up to set seed and the progeny (T2 seedlings) will be screened for phenotypic and cell death response defects, as described for the insertional mutants above. Several transgenic lines (10 or more) will be characterized to identify a common defective phenotype or response. Phenotypic response will be related to the level of mRNA of the gene of interest accumulated in individual RNAi lines, as measured by semi-quantitative RT-PCR or quantitative real-time PCR. The expectation would be that the more severe the defective phenotype, the lower the abundance of the mRNA for the gene targeted by RNAi.

APPENDIX 1
Statistical data for individual protein spots

| Master # | Accession | | Glc-Hexokinase | |
|---|---|---|---|---|
| (Hk) | Number | Protein name | Ratio | P-value |
| 613 | H84453 | Probable heat shock protein [imported] | | |
| 795 | gi15232682 | Heat shock protein hsc70-3 | | |
| 775 740 | 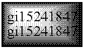 | Heat shock cognate 70 kDa protein 2 (hsc70-2) Heat shock cognate 70 kDa protein 2 (hsc70-2) | | |
| 778 | gi1072473 | dnaK-type molecular chaperone hsc70.1 | | |
| 906 | gi15219234 | ATPase 70 kDa subunit-related protein | 1.21 | 0.035 |
| 1205 | gi2493132 | Vacuolar ATP synthase subunit B isoform 2 | 1.09 | 0.05 |
| 1229 1245 | 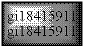 | H+-transporting ATP synthase beta chain H+-transporting ATP synthase beta chain | | |
| 1196 | gi14916970 | ATP synthase alpha chain, mitochondrial | | |
| 707 706 703 | 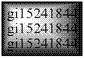 | Luminal binding protein 1 precursor (BiP-1) Luminal binding protein 1 precursor (BiP-1) Luminal binding protein 1 precursor (BiP-1) | | |
| 699 | gi30693966 | Luminal binding protein 2 (BiP-2) | | |
| 2572 | gi15224581 | Putative glutathione S-transferase | −1.16 | 0.029 |
| 2562 | gi15224582 | Putative glutathione S-transferase | −1.29 | 0.0031 |
| 2592 2539 | 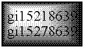 | Putative glutathione S-transferase Putative glutathione S-transferase | 1.11 1.15 | 0.028 0.0098 |
| 2575 2602 2591 | 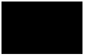 | Putative glutathione S-transferase Putative glutathione S-transferase Putative glutathione S-transferase | 1.96 2.98 3.01 | 0.012 0.0055 0.0065 |
| 2579 | gi2554769 | Chain A, Structure Of glutathione S-transferase | | |
| 2690 | gi20138157 | Phospholipid hydroperoxide glutathione peroxidase | | |
| 2552 2443 | 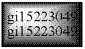 | L-ascorbate peroxidase 1, cytosolic (APX1) L-ascorbate peroxidase 1, cytosolic (APX1) | | |
| 1525 | 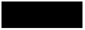 | Putative S-adenosylmethionine synthetase | −1.37 | 0.0009 |
| 1516 | 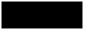 | Putative S-adenosylmethionine synthetase | −1.37 | 0.01 |
| 1481 | gi15234354 | S-adenosylmethionine synthetase 2 | −1.08 | 0.042 |
| 1282 1292 | 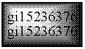 | Adenosylhomocysteinase Adenosylhomocysteinase | | |
| 1241 1257 1264 | 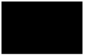 | Enolase (2-phospho-D-glycerate hydrolase) Enolase (2-phospho-D-glycerate hydrolase) Enolase (2-phospho-D-glycerate hydrolase) | 1.15 | 0.05 |
| 1817 | gi3435286 | GAPDH C subunit (GapC) | | |
| 1748 | gi21593240 | GAPDH C subunit (GapC) | 1.19 | 0.013 |
| 1254 | gi15227559 | Tubulin beta-7 chain | 1.09 | 0.034 |
| 1200 | gi15241168 | Tubulin alpha-3/alpha-5 chain (TUA3) | −1.66 | 0.0041 |
| 1242 1238 | 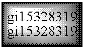 | Putative mitochondrial aldehyde dehydrogenase Putative mitochondrial aldehyde dehydrogenase | 1.25 1.29 | 0.0013 0.014 |
| 1138 | gi21954069 | Putative alanine aminotransferase | 1.65 | 0.05 |
| 1187 | gi21954071 | Putative alanine aminotransferase | 1.3 | 0.00073 |
| 1135 | gi9082270 | Alanine aminotransferase | 1.24 | 0.0071 |
| 1069 | gi15232827 | Threonine ammonia lyase | 1.17 | 0.049 |
| 1718 | gi15229530 | Glutamine synthetase-related protein | −1.38 | 0.0039 |
| 482 | gi11358581 | nodulin/glutamate-ammonia ligase-like protein | 1.42 | 0.003 |
| 1959 1955 | 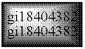 | Malate dehydrogenase [NAD], mitochondrial Malate dehydrogenase [NAD], mitochondrial | 1.27 1.18 | 0.0034 0.005 |
| 1964 | gi15232468 | Malate dehydrogenase [NAD], mitochondrial | 1.09 | 0.016 |
| 1943 | gi25283601 | Malate dehydrogenase [EC 1.1.1.37], chloroplast | 1.24 | 0.00065 |
| 690 | gi30693102 | NADH dehydrogenase (ubiquinone), mitochondrial | 1.19 | 0.049 |
| 1892 | gi14334740 | Putative fructose-bisphosphate aldolase | 1.1 | 0.012 |
| 1868 | gi15226185 | Putative fructose-bisphosphate aldolase | | |
| 2370 | gi18390982 | ATP-dependent Clp protease proteolytic subunit | −1.71 | 0.0075 |
| 2422 | gi20260140 | Multicatalytic endopeptidase complex alpha subunit | | |
| 2626 | gi15228805 | 20S proteasome beta subunit D (PBD1) | | |
| 1098 | gi288063 | Ketol-acid reductoisomerase | 1.23 | 0.028 |
| 1074 | gi11094242 | Cytosolic phosphoglucose isomerase | | |
| 1155 | gi30684419 | Dihydrolipoamide dehydrogenase 2, mitochondrial | 1.1 | 0.014 |
| 1649 | gi21593388 | 12-oxophytodienoate reductase (OPR1) | | |
| 1920 | gi15237888 | Putative NADP-dependent oxidoreductase (P1) | −1.2 | 0.015 |
| 1148 | gi21593375 | Unknown protein | 1.17 | 0.028 |
| 1460 | gi7485313 | Hypothetical protein F10M10.120 | | |
| 2734 | gi18412406 | Expressed protein | | |
| 2200 | gi15227803 | Expressed protein | −1.26 | 0.021 |
| 2282 | gi15228883 | Expressed protein | −1.29 | 0.0031 |
| 2221 | gi21593226 | Putative esterase D | −1.16 | 0.0032 |
| 2832 | gi30697295 | Actin depolymerizing factor 3-like prptein | | |
| 2842 | gi15218373 | 40S ribosomal protein S12 (RPS12A) | 1.15 | 0.021 |
| 1063 | gi30687521 | Putative protein disulfide isomerase | 1.21 | 0.044 |
| 1281 | gi15234516 | Putative thioredoxin | −1.18 | 0.041 |
| 2605 | gi15239652 | Putative quinone reductase | 1.69 | 0.00037 |
| 1439 | gi14594802 | Translation initiation factor elf-4A1 | | |
| 1497 | gi21536853 | Putative phosphoglycerate kinase | −1.41 | 0.029 |

| Master # | AMP-PCP | | F-400 | | ATP treatment | | AF-400/F-400 | |
|---|---|---|---|---|---|---|---|---|
| (Hk) | Ratio | P-value | Ratio | P-value | Ratio | P-value | Ratio | P-value |
| 613 | −1.26 | 0.041 | −2.12 | 0.015 | | | | |
| 795 | 1.2 | 0.0089 | −1.51 | 0.0054 | 1.26 | 0.014 | | |
| 775 | | | −2.18 | 0.0015 | | | | |
| 740 | −1.22 | 0.0029 | −1.8 | 0.00037 | 1.66 | 0.013 | 1.52 | 0.015 |
| 778 | 1.15 | 0.031 | −2 | 0.0018 | 1.24 | 0.05 | | |
| 906 | 1.19 | 0.0032 | | | | | | |
| 1205 | 1.24 | 0.003 | 1.25 | 0.039 | −1.14 | 0.047 | −1.17 | 0.02 |
| 1229 | 1.4 | 0.005 | 1.32 | 0.019 | | | | |
| 1245 | 1.23 | 0.0028 | 1.24 | 0.017 | −1.19 | 0.0071 | | |
| 1196 | 1.31 | 0.0017 | 1.33 | 0.014 | | | | |
| 707 | 1.45 | 0.018 | −2.68 | 0.00066 | 1.94 | 0.0057 | | |
| 706 | | | −2.88 | 0.00064 | 2.9 | 0.024 | 1.46 | 0.039 |
| 703 | 1.29 | 0.0021 | −1.35 | 0.035 | 1.51 | 0.0025 | | |
| 699 | | | −2.5 | 0.0045 | 2.61 | 0.023 | | |
| 2572 | | | 3.33 | 0.0055 | −1.28 | 0.016 | | |
| 2562 | 1.4 | 0.0092 | | | −1.6 | 0.0033 | | |
| 2592 | | | 5.88 | 0.0017 | | | | |
| 2539 | | | 36.51 | 0.0004 | | | −1.39 | 0.033 |
| 2575 | | | 4.57 | 0.0022 | | | | |
| 2602 | | | 5.43 | 0.00025 | | | | |
| 2591 | | | 5.59 | 0.00015 | | | | |
| 2579 | | | 20.84 | 4.5e-005 | | | | |
| 2690 | 1.21 | 0.0025 | 1.96 | 0.0033 | | | | |
| 2552 | −1.47 | 0.0017 | | | 1.19 | 0.01 | | |
| 2443 | −2.51 | 0.00012 | −2.02 | 0.037 | 1.41 | 0.025 | 1.98 | 0.035 |
| 1525 | | | | | 1.14 | 0.04 | | |
| 1516 | 1.22 | 0.04 | | | | | | |
| 1481 | −1.3 | 0.0016 | | | 1.2 | 0.028 | | |
| 1282 | 1.46 | 0.00024 | −1.56 | 0.0077 | −1.25 | 0.016 | | |
| 1292 | 1.29 | 0.0014 | −1.14 | 0.0065 | −1.36 | 0.0099 | | |
| 1241 | 1.39 | 0.0018 | 1.13 | 0.016 | | | | |
| 1257 | 1.51 | 0.00024 | | | −1.3 | 0.0061 | | |
| 1264 | 1.32 | 0.0037 | | | −1.34 | 0.0015 | | |
| 1817 | 1.13 | 0.0064 | | | −1.2 | 0.0073 | | |
| 1748 | 1.21 | 0.0021 | | | −1.16 | 0.0062 | | |
| 1254 | −1.2 | 0.049 | | | | | | |
| 1200 | 1.35 | 0.0013 | | | −1.23 | 0.0058 | | |
| 1242 | | | 1.53 | 0.04 | −1.19 | 0.0096 | | |
| 1238 | | | 2.32 | 0.0021 | | | | |
| 1138 | | | | | | | | |
| 1187 | 1.41 | 0.0004 | 1.42 | 0.0083 | | | | |
| 1135 | 1.2 | 0.021 | | | | | | |
| 1069 | −1.23 | 0.041 | −1.22 | 0.049 | −1.28 | 0.0017 | | |
| 1718 | −1.13 | 0.011 | | | | | | |
| 482 | | | | | −1.29 | 0.0053 | | |
| 1959 | −1.11 | 0.0054 | | | 1.24 | 0.046 | | |
| 1955 | | | | | −1.16 | 0.0044 | | |
| 1964 | 1.21 | 0.021 | 1.18 | 0.0087 | | | | |
| 1943 | 1.22 | 0.0041 | 1.32 | 0.017 | | | | |
| 690 | −1.44 | 0.00015 | | | | | | |
| 1892 | −1.71 | 0.00088 | | | | | | |
| 1868 | −1.35 | 0.0027 | | | | | | |
| 2370 | 1.38 | 0.0078 | | | 1.4 | 0.021 | | |
| 2422 | −1.21 | 0.0067 | 1.19 | 0.016 | 1.23 | 2.2e-005 | | |
| 2626 | 1.14 | 0.0049 | 1.84 | 3.7e-005 | | | | |
| 1098 | −1.12 | 0.0051 | | | | | | |
| 1074 | 1.2 | 0.001 | 1.34 | 2.5e-005 | −1.22 | 0.012 | | |
| 1155 | 1.34 | 0.0011 | | | −1.14 | 0.027 | | |
| 1649 | −1.22 | 0.0039 | 1.35 | 0.028 | −1.55 | 0.019 | −1.25 | 0.0053 |
| 1920 | −1.45 | 0.00011 | 1.74 | 0.014 | | | | |
| 1148 | 1.24 | 0.0046 | | | | | | |
| 1460 | −1.1 | 0.022 | | | −1.21 | 0.003 | | |
| 2734 | −1.93 | 5.7e-005 | −1.98 | 0.001 | 1.42 | 0.01 | 1.41 | 0.032 |
| 2200 | | | −1.33 | 0.023 | −1.22 | 0.01 | | |
| 2282 | −1.74 | 0.00065 | | | | | | |
| 2221 | −1.24 | 0.0093 | −1.14 | 0.0059 | | | | |
| 2832 | −1.29 | 0.0011 | | | 1.29 | 1.1e-005 | | |
| 2842 | −1.3 | 0.005 | | | 1.2 | 0.0035 | | |
| 1063 | 1.24 | 0.003 | 1.45 | 0.021 | | | | |
| 1281 | 1.23 | 0.0011 | −1.74 | 0.0014 | 1.19 | 0.0035 | 1.35 | 0.0068 |
| 2605 | 1.38 | 0.0058 | 4.45 | 3.1e-005 | | | | |
| 1439 | 1.84 | 0.0098 | 1.19 | 0.018 | | | | |
| 1497 | 1.47 | 0.019 | 1.13 | 0.046 | −1.16 | 0.034 | | |

(Grey and black blocks highlight adjacent protein spots with the same gene identity)
Ratio = abundance of spot in treatment divided by abundance of spot in control
F-400 = Fusarium elicitor treatment
AF-400 = Treatment with Fusarium elicitor mixed with ATP
AF-400/F-400 = Comparison between ATP+F-400 treatment with F-400 only treatment. The ratio here refers to AF-400 divided by F-400.

1. A method of controlling the viability of a plant cell or cells by contacting the plant cell or cells with a substance which directly or indirectly up- or down-regulates a cell death pathway in the cell or cells, which pathway is activatable by depleting the concentration of NTP in the external environment available to the cell or cells whose viability is to be controlled.

2. A method according to claim 1, wherein the pathway is activatable by depleting the concentration of ATP in the external environment available to the cell or cells.

3. A method of killing a plant cell or plant cells according to claim 1 by activating a cell death pathway, which pathway is activatable by depletion of extracellular NTP, especially ATP, available to the cell for utilization.

4. A method according to claim 3, wherein the plant cell or cells are present in a plant and performance of the method results in death of the plant or a part thereof.

5. A method according to claim 3 wherein the cell death pathway is activated by depletion of extracellular ATP available to the cell for utilization.

6. A method according to claim 3 comprising the step of bringing an active agent into contact with the extracellular environment of the cell or cells to be killed, wherein the agent hydrolyses extracellular NTP (especially ATP) and/or renders extracellular NTP (especially ATP) non-available to the plant cell or cells.

7. A method according to claim 1 comprising the use of an NTP (especially an ATP) analogue and/or an ATPase inhibitor.

8. A method according to claim 7, wherein the NTP analogue is substantially non-hydrolysable by the plant extracellular NTPase (especially ATPase) enzymes.

9. A method according to claim 1 comprising the use of a non-hydrolysable NTP analogue selected from the group consisting of: AMP-PCP, AMP-PNP, ATP-γ-S, GMP-PCP, GMP-PNP and GTP-γ-S.

10. A composition for controlling the viability of a plant cell or plant cells, the composition comprising an active agent which, directly or indirectly, up- or down-regulates in the cell or cells a cell death pathway, which pathway is activatable by depletion of extracellular NTP (especially ATP) available for utilization by the cell or cells.

11. A composition according to claim 10 comprising an inhibitor or antagonist of one or more of the polypeptides identified in Table 3 herein.

12. A composition according to claim 10, wherein the active agent is a substance which activates the cell death pathway.

13. A composition for controlling the viability of a plant cell or plant cells, the composition comprising an active agent which, directly or indirectly, up- or down-regulates in the cell or cells a cell death pathway, which pathway is activatable by depletion of extracellular NTP (especially ATP) available for utilization by the cell or cells for use in the method of claim 1.

14. A composition according to claim 10 further comprising one or more components of conventional herbicidal compositions, selected from the group consisting of surfactants and penetration enhancers.

15. (canceled)

16. (canceled)

17. A method of preserving the viability of a plant cell or cells exposed to viability-threatening extracellular NTP depletion, the method comprising the step of administering a viability-preserving substance which has the effect, directly or indirectly, of increasing the extracellular NTP (especially ATP) concentration or otherwise inhibiting the cell death pathway which is activatable by depletion of extracellular NTP (especially ATP) available to the cells.

18. A method according to claim 17, wherein the viability-preserving substance comprises a nucleotide sequence and/or the polypeptide(s) encoded thereby, the nucleotide sequence encoding a polypeptide which, when expressed in the plant cell, has the effect of tending to increase the extracellular concentration of NTP (especially ATP) so as to preserve the viability of the cell.

19. A method according to claim 18, wherein the nucleotide sequence is expressed constitutively in the plant cell.

20. A method according to claim 18, wherein the nucleotide sequence is expressed in the plant cell in response to a viability-threatening event.

21. A method of altering the viability of a plant or part thereof, the method comprising the step of introducing into the plant or part thereof a recombinant nucleic acid molecule comprising a sequence of at least 200 bases (preferably at least 300 bases, more preferably at least 400 bases) having at least 90% sequence identity with a sequence encoding one of the polypeptides listed in Table 3, operably linked in the sense or antisense orientation to a promoter active in a plant.

22. A method according to claim 21, comprising the use of two or more different sequences, each of at least 200 bases, each having at least 90% sequence identity with a respective different sequence encoding one of the polypeptides listed in Table 3.

23. A method according to claim 22, wherein each of the two or more sequences is present on a single nucleic acid construct.

24. A method according to claim 21 wherein the promoter is inducible and/or a tissue-specific promoter.

25. A method according to claim 21 wherein the introduced molecule directs the expression of an active polypeptide, so as to increase the concentration of the polypeptide (optionally in response to one or more specific stimuli) in the plant or part thereof.

26. A method according to claim 21 wherein the introduced molecule directs the synthesis of a transcript which has an inhibitory effect (for example, as a result of antisense or RNAi interactions) on the expression of an endogenous gene present in the plant.

27. Use of a nucleic acid sequence as aforesaid in the preparation of a composition for the method of claim 21.

28. A transgenic plant, or the progeny thereof, produced by the method of claim 21.

29. Use of an ATP analogue and/or an ATPase inhibitor as an active agent in the preparation of a herbicidal composition.

30. A use according to claim 29, wherein the ATP analogue is non-hydrolysable.

31. Use of an inhibitor of any one of the polypeptides identified in Table 3 as an active agent in the preparation of a composition to alter the viability of a plant.

32. Use according to claim 31, wherein the composition is a herbicidal composition.

33. Use according to claim 31, wherein the composition is a viability-preserving composition.
